(19) United States
(12) Patent Application Publication
(10) Pub. No.: US 2008/0269549 A1
(43) Pub. Date: Oct. 30, 2008

(54) METHOD, ARTICLE, AND APPARATUS FOR CRYOPRESERVATION OF BIOLOGICAL SAMPLES

(75) Inventors: Robert Taft, Southwest Harbor, ME (US); Shannon Byers, Lamoine, ME (US); Jane Farley, Ellsworth, ME (US); Jeffrey Forthofer, Southwest Harbor, ME (US); Joan Malcolm, Sullivan, ME (US); Michael Wiles, Mount Desert, ME (US)

Correspondence Address: JAGTIANI + GUTTAG, 10363-A DEMOCRACY LANE, FAIRFAX, VA, 22030, US

(73) Assignee: The Jackson Laboratory, Bar Harbor, ME (US)

(21) Appl. No.: 12/108,727

(22) Filed: Apr. 24, 2008

Related U.S. Application Data
(60) Provisional application No. 60/926,521, filed on Apr. 27, 2007.

(51) Int. Cl. A61B 17/43; A01N 1/02; C12M 1/00; A61M 1/00

(52) U.S. Cl. 600/35; 62/64; 435/307.1; 604/319

(57) ABSTRACT

Described herein is a method of storing a biological sample, such as a sperm sample, in an elongated container for cryopreservation. Multiple volumes of biological sample may be placed in each elongated container such that each volume of sample is separated from its nearest neighbor by a separation gas. The present invention further relates to an apparatus and method for loading biological samples, such as sperm samples, into an elongated container for cryopreservation of the sample material. The apparatus and method employs drawing the sample into the elongated container such that individual sample volumes are interspaced by gaps of separation gas. This alternating pattern may be created by repeatedly contacting and removing the elongated container from the sample under suction pressure. The apparatus further incorporates the use of a computer to control the volume and number of aliquots as desired. A container according to such methods is also provided.

### CROSS-REFERENCE TO RELATED APPLICATIONS

This application claims the priority benefit of U.S. Provisional Patent App. No. 60/926,521 filed on Apr. 27, 2007, the disclosure of which is hereby incorporated by reference.

### GOVERNMENT INTEREST STATEMENT

The invention claimed in this application may have been arrived at by work supported, at least in part, by funds pursuant to Grant Number RR01262-24, awarded by the National Institutes of Health. The government may have rights in this patent application pursuant to such grant.

### BACKGROUND

1. Field of the Invention

The present invention relates to an apparatus and method for loading solutions of biological samples into an elongated container for cryopreservation of the sample material as well as the elongated container itself containing such biological samples, particularly samples containing sperm or cells, for storage and cryopreservation. The present invention further relates to methods of generating live offspring from sample material cryogenically preserved according to present methods.

2. Related Art

Cryopreservation of biological materials, tissues and cells has numerous research, clinical, and veterinary applications. For example, cryopreservation may reduce the expense and facility space for maintaining live breeding colonies of plants or animals as well as live cultures of cells and tissues that may have future experimental or clinical value. According to some clinical or veterinary applications, biological samples may be preserved for later use in treatments or therapies. In particular, cryopreservation may be applied to treatments and experiments relating to fertility and fertilization by allowing gametes, including sperm and oocytes, to be preserved for later use.

Revolutionary advances in genome research and the ability to create genetically specific strains of mice have resulted in an exponential increase in the number of mouse strains available for biomedical research. These newly created strains, particularly those in which the strain or mutation can be maintained by haploid germplasm, are most efficiently preserved by freezing and storing sperm. For example, the sperm from a single male mouse can theoretically generate several thousand progeny. However, existing methods to collect and freeze sperm from a single male mouse in several straws with each straw containing one droplet of sperm sample yielding an average of 10-15 live born mice per straw. The sperm from a single male mouse can theoretically generate several thousand progeny. However, to do so, it would be necessary to freeze sperm in 150-200 straws to yield 1500 to 3000 live offspring. A need exists for new devices and methods that increase the efficiency of cryopreservation and recovery of sperm and allow the rapid expansion of small animal strains, such as mice. Furthermore, new devices and methods are needed for the cryopreservation and storage of other biological materials, including cells and tissues and particularly stem cells, to improve their storage and recovery efficiency.

With regard to existing devices and techniques, straws are typically filled with a biological sample (e.g., a sperm sample). However, these available devices and techniques have several disadvantages and limitations. First, hand pipetting samples into the tubes or straws is time and labor intensive, is not ergonomic for the user, and is more prone to error (i.e., less accurate). Second, many straw loading devices first draw the sample into the apparatus itself before continuing to fill or load each of the straws with the sample. As a result, there is the potential for cross-contamination of samples with these existing devices and techniques if the internal tubing and other surfaces of the device that contact the sample are not cleaned and/or replaced between applications.

Finally, existing straw loaders are generally bulk loaders that fill individual straws with a single large volume of sample. Although this may be sufficient in some circumstances (e.g., agriculture purposes involving larger animals), they are generally not suitable for use with much smaller sample sizes that are often generated from smaller animal sources including many research animals (e.g., rats, mice, etc.). Loading substantial volumes of sample into straws or tubes with existing devices and techniques also has the drawback of losing some of the sample to “dead volume” as a result of the instrumentation aspiration requirements. In addition, existing devices and techniques lack the adjustability, accuracy, and precision that are necessary for tailoring their use under a variety of circumstances based on the sample type and size, the species source, and the intended application for the sample following storage.

Therefore, a further need exists in the art for improved methods and devices for loading a sample of biological material (e.g., a solution containing sperm) into a container for cryopreservation in a manner that is adjustable, automated, and accurate, that is suitable for loading smaller sample volumes, and that improves storage capacity and increases the number of offspring per straw while avoiding internal cross-contamination between separate applications and/or samples as with existing devices and techniques.

### SUMMARY

According to one broad aspect of the invention, a method of storing a biological sample in an elongated container, such as a tube or straw, is provided. As a result, smaller individual aliquots of sample are stored in each elongated container with each separated from its nearest neighbor(s) by a volume of separation gas. Surprisingly, when such method is used to store sperm, significantly more live born animals result after thawing and recovery than result from using the conventional approach of storing a single volume of sperm in multiple containers or straws. Such method may be used to store sperm obtained from one animal, such as one mouse, in an elongated container, or such method may be used to store sperm obtained from multiple animals, such as more than one mouse, in each elongated container. By using the method of the present invention to cryopreserve sperm, it is possible to rapidly increase the number of animals (e.g., rapidly expand the size of a mouse colony) from fewer elongated containers without the need for breeding. In addition, biological samples of cells and/or tissues, including sperm, may be stored more efficiently at a higher density in each elongated container, thus requiring less storage space.

According to some embodiments, the biological sample is stored in a cryoprotective media (CPM). Each volume or droplet of biological sample in CPM within the elongated container is separated from its respective nearest neighbor(s) by a volume of separation gas. Such elongated container containing such biological sample may be further cryopreserved.

According to another broad aspect of the present invention, an elongated container containing two or more cryopreserved volume(s) or droplet(s) of biological sample is described, wherein each volume or droplet of biological sample is separated from each of its respective nearest neighbor(s) by a volume of separation gas. Such elongated container may be further cryopreserved.

According to yet another aspect of the present invention, an apparatus is provided with means for loading two or more aliquots of a biological sample into an elongated container. The apparatus raises and lowers the distal end of the tube via a drive system such that it undergoes repeated cycles of contact with and withdrawal from the sample solution. The elongated container is under suction pressure generated by a suction system connected to a proximal end of the elongated container such that an alternating pattern of sample and separation gas is drawn into the elongated container as its distal end oscillates between contact with and withdrawal from the sample. Thus, each aliquot or droplet of sample that is drawn into the elongated container is divided and separated by an interposed gap of separation gas. Unlike existing devices, the tube itself (that is eventually cut, sealed, and used for storage and/or freezing of the sample) directly contacts the sample solution, thus drawing the sample directly into the tube without first passing the sample through any additional tubing or surfaces that may be present within the device.

Alternatively, the distal end of an elongated container of the apparatus may be connected to a pre-cut straw, which is itself put in direct contact with the sample. Again, the apparatus raises and lowers the straw connected to the elongated container such that it undergoes repeated cycles of contact and withdrawal from the sample solution. Since the straw is under suction pressure via the elongated container and suction system, an alternating pattern of sample and air is drawn into the straw as it oscillates between contact with and withdrawal from the sample, thus dividing and separating each aliquot or droplet of sample that is drawn into the straw by an interposed gap of separation gas.

According to another aspect of the invention, the apparatus may alternatively raise and lower the vessel containing the sample to allow the straw or distal end of the elongated container to contact with and withdrawal from the sample. In any case, the apparatus may be further connected to a computer system running a software program to automate the operation of the apparatus. Accordingly, the computer system and associated software may control the rate and/or timing of each cycle as well as the amount of suction pressure generated by the suction system. Thus, the computer and its associated software allow for accurate and precise control of the amounts of sample that are loaded into the elongated container for each sample and may be programmed to perform the loading procedure appropriately depending on the specific application and sample source. By increasing the accuracy and precision of sample aliquots and air/gas volumes drawn into the elongated container, more sample aliquots may also be drawn into a given length of each elongated container with less risk of intermixing sample aliquots.

The sample itself is a suspended biological sample that is capable of being drawn into an elongated container. Once the desired number of sample aliquots of appropriate volume is loaded into an elongated container, the elongated container may be cut and/or sealed on either or both ends of the length of the elongated container containing the sample aliquots so that the sealed length or segment of elongated container may be frozen and/or stored for later use.

According to another broad aspect of the invention, methods for generating a large cohort of animals as well as live animals or offspring are described using methods of the present invention.

### BRIEF DESCRIPTION OF THE DRAWINGS

The invention will be described in conjunction with the accompanying drawings, in which:

FIG. 1 shows one embodiment of the apparatus for loading an elongated container with a biological sample;

FIG. 2 is a flowchart showing a method of operation for the invention according to some embodiments for loading sample into an elongated container;

FIG. 3 shows an alternative embodiment of the apparatus for loading a straw with a biological sample;

FIG. 4 is a flowchart showing an alternative method of operation for the invention according to some embodiments for loading sample into a straw;

FIG. 5 shows the rate of fertility depending on the volume of sperm sample frozen;

FIG. 6 shows the rate of fertility depending on the number of sperm samples frozen;

FIG. 7 shows a comparison of fertilization rates between one large volume and multiple smaller volumes of frozen sperm samples; and

FIG. 8 shows the rate of fertility for a range of freeze volumes of sperm sample.

### DETAILED DESCRIPTION OF THE PREFERRED EMBODIMENTS

### Definitions

It is advantageous to define several terms before describing the invention. It should be appreciated that the following definitions are used throughout this application.

For the purposes of the present invention, the term “distal” refers to the end of an elongated container positioned closest to a vessel containing a biological sample.

For the purposes of the present invention, the term “proximal” refers to the end of an elongated container opposite to the distal end.

For the purposes of the present invention, the term “separation gas” refers to any gas that may be used to separate two nearest neighbors of biological samples of the present invention. Suitable gases that may be used as a separation gas may include air, noble gases such as helium, neon, argon, etc., nitrogen, etc.

For the purposes of the present invention, the terms “nearest neighbor” or “nearest neighbors” or “nearest neighbor(s)” refer to one or more volumes of biological sample, such as a sperm-CPM or cell-CPM sample, that are separated from another volume of biological sample in an elongated container only by a volume of separation gas.

For the purposes of the present invention, the term “elongated container” refers to any container that has a length that is significantly larger than its diameter or its breadth and width. An elongated container may have any cross-section shape such as circular, ellipsoid, square, rectangular, hexagonal, octagonal, etc. The diameter, breadth, width, etc. of an elongated container may vary over the length of the container Examples of elongated containers include tubes, straws, etc. An “elongated container” may be rigid or flexible and may be made of any polymeric or plastic material that may be suitable for being sealed and/or frozen for cryopreservation such as, for example, a PETG or PVC material.

For the purposes of the present invention, the term “straw” may be any pre-cut length of tubular material in any shape and/or cross-section. A “straw” may be rigid or flexible and may be made of any polymeric or plastic material that may be suitable for being sealed and/or frozen for cryopreservation. A “straw” may include any pre-cut tubular material that is currently known, used, and/or understood in the art for use as a straw in cryopreservation of any type of biological sample. For example, a “straw” may include any straw that is commercially available. A “straw” may contain any volume of sample and include, for example, a 250 microliter straw (IMV), a 300 or 500 microliter straw or French straw (CryoBioSystem), or a 2 ml straw, the latter of which may be suitable for some samples of cells or tissues. A “straw” may be preloaded with a solution, and/or it may contain a cotton swab or plug or other porous material. A “straw” may further include the term “mini-straw” as commonly used.

For the purposes of the present invention, the term “unibody construction” refers to an article made from a single piece of material, such as a tube or straw.

The terms “sample” and “biological sample” refer to a solution containing a biological material. At least a portion of the biological material is suspended in a solution such that the solution containing the biological material may be freely placed in an elongated container without causing blocking or hindering the introduction of additional sample and/or separation gas into the elongated container. The “solution” used may be any conventional or known solution or media used for cryopreservation of biological samples. The term “solution” may further include, for example, cryoprotective media (CPM) as described below as well as in U.S. Utility application Ser. No. 11/811,968 and U.S. Provisional Application Nos. 60/926,521, 60/812,833, 60/840,744, and 60/854,501, the disclosures of which are hereby incorporated by reference.

The term “biological material” includes any tissue, culture, cells, and/or gametes, or any combination thereof, that are capable of being placed in an elongated container. The tissues, cells, and/or gametes may be from any species source but are preferably from a mammalian and/or vertebrate species. According to one embodiment, the biological material contains sperm. According to other embodiments, the biological material contains embryos, oocytes, tissue(s), cultures, or cells (e.g., primary cells, stem cells, etc.).

The term “sperm” is intended to mean a cell or specialized cell at any stage of sperm development or spermatogenesis, including, for example, spermatocytes, spermatogonia, spermatozoa, spermatids, immature and mature sperm, etc.

For the purposes of the present invention, the term “sperm-CPM” sample refers to a composition comprising sperm in a cryoprotective medium (CPM).

For the purposes of the present invention, the term “cell-CPM” sample refers to a composition comprising cells in a cryoprotective medium (CPM).

The terms “mammal” and “mammalian” are intended to include any animal classified phylogenetically or generally understood to be a mammal, including, but not limited to, humans, monkeys, and other primates. The terms “mammal” and “mammalian” also refer to animals having an agricultural or domesticated use or purpose including, but not limited to, cattle, sheep, goats, pigs, horses, canines, cats, etc., as well as animals having research or laboratory uses including, but not limited to, rabbits, mice, rats, etc. The terms “mammal” and “mammalian” further include any wild-type, mutated, or transgenic mammal as well as any genetic lines of mammals.

The terms “vertebrate” or “vertebrate species” are intended to include any animal classified phylogenetically or generally understood to be a vertebrate, including, but not limited to mammalian and non-mammalian species including birds, reptiles, etc. The terms “vertebrate” or “vertebrate species” further include any wild-type, mutated, or transgenic vertebrate as well as any genetic lines of vertebrates.

For purposes of present invention, the term “multiport manifold” refers to a device for splitting a channel or lumen of a first elongated container or tube into multiple channels or lumens such that a second plurality of elongated containers may be attached to the first elongated container via the multiport manifold. For example, such multiport manifold of the present invention may be used to attach multiple pre-cut straws and/or tubes to another tube connected to the suction system. In this sense, multiport manifold is able to deliver suction pressure generated by the suction system to the multiple pre-cut straws and/or tubes connected to the multiport manifold. Such multiport manifold allows multiple straws and/or tubes to be filled with sample and/or separation gas together or in unison.

### Description

According to some aspects of the present invention, a method is provided for storing a biological sample or set of biological samples in an elongated container for cryopreservation. By separating individual aliquots or droplets of sample by a volume or gap of separation gas, multiple samples may be stored in each elongated container. Thus, by increasing the density of samples per elongated container, instead of in multiple containers, the storage efficiency for the biological sample(s) is improved since less storage space is consumed. Furthermore, as described below, greater fertilization efficiency per volume is achieved when smaller volumes of sperm are frozen. This improved fertilization efficiency continued even when multiple sperm samples of small volume are separated from their nearest respective neighbor(s) by volume(s) of separation gas within the elongated container. In addition to the benefit of improved storage efficiency, it remains possible that other types of biological tissue(s), culture(s), cell(s) or gamete(s) may also see improved viability and/or usefulness after thawing using the methods of the present invention.

With respect to cryopreservation of sperm, there is substantial utility in being able to generate a large cohort of animals, such as mice, using a single elongated container of frozen sperm, in addition to the benefit of increased storage capacity. For example, pharmacological experiments and screens often require large cohorts of age-matched mice having the same genotype. If these mice need to be generated from cryopreserved strains using currently available methods, either several straws or several rounds of mating are needed to create a sufficient number of mice. By contrast, methods of the present invention as described herein allow for more progeny to be produced from each elongated container than result from presently available methods. This is due in part to the increased viability and fertilization efficiency achieved after thawing when smaller volumes of sperm sample are frozen compared to a single large volume. Furthermore, the same benefit is achieved when multiple sperm samples are stored in each elongated container and separated from each other by volume(s) of separation gas. As a result, fewer resources are consumed in rapidly expanding a mouse colony from a sample of sperm, and there is the additional benefit that a smaller fraction of the total cryopreserved stock of sperm sample stored in elongated containers may be needed to generate a sufficient number of age- and sex-matched animals.

Surprisingly, when a smaller volume of sperm sample in CPM is frozen, an increased efficiency of fertilization per volume of sperm is produced compared to larger volumes of frozen sperm samples. For example, a high fertilization rate results when droplets of from about 5 microliter to about 30 microliter are cryopreserved in a 250 microliter French straw. In contrast, a significantly lower fertilization rate is observed when one droplet of 100 microliter is cryopreserved in 250 microliter French straws. According to the set of experiments shown in Example 1 (below), when a single small volume of mouse sperm (i.e., about 10 microliters) per straw was frozen, the fertilization rate (measured as a percentage of oocytes developing to 2-cell stage embryos) was about 45%. In contrast, when a single larger volume of mouse sperm (i.e., about 100 microliters) per straw was frozen, a lower fertilization rate of about 18% was observed. Typically, mouse sperm is present in CPM droplets within the range of from about 5×10³ per microliter to 5×10⁵ sperm per microliter. In one embodiment, sperm is present in one 10-microliter CPM droplet at 3×10⁵ sperm. See also Example 4 (below) demonstrating the relationship between fertilization efficiency of sperm after thawing over a range of freeze volumes in each straw.

This improved fertilization efficiency that is observed with smaller volumes remains when straws are filled with several small volumes or droplets of sperm-CPM separated from their nearest neighbor(s) by a gap of air. According to the set of experiments in Example 2 (below), when four 10 microliter sperm samples were frozen in each straw, the fertilization rate (measured as a percentage of oocytes developing to 2-cell stage embryos) after thawing was roughly the same as when one 10 microliter sample (both about 51%). Therefore, it is feasible to place up to ten or more aliquots or droplets of 5-10 microliters sperm-CPM in one straw, such as a 250 microliter (0.25 ml) French straw (IMV, Maple Grove, Minn.), without sacrificing the improved fertilization efficiency with smaller sample volumes. As also shown in separate experiments in Example 3 (below), the fertilization efficiency is higher when 10 volumes of sperm (10 microliters each) were frozen compared to a single large volume of sperm (100 microliters).

Therefore, it is feasible that a larger number of offspring are capable of being generated from each elongated container when multiple sperm samples of small volume separated by a gap of separation gas are frozen in each elongated container due to the improved fertilization efficiency and the ability to store multiple aliquots or droplets of sample in each elongated container. In the method of this invention, two or more isolated droplets or aliquots of sperm-CPM are transferred into each elongated container, in which each droplet is separated from it respective nearest neighbor(s) by a volume of separation gas. The number of sample aliquots placed into each elongated container depends on the dimensions of the elongated container used and/or the size of the sample aliquots. For example, 2 or more droplets may be placed in one 250 microliter straw. The volume of a droplet may be from about 5 microliters to about 50 microliters, such as a volume of from about 5 microliters to about 40 microliters, a volume of from about 5 microliters to about 30 microliters, or in particular embodiments, a volume from about 5 microliters to about 10 microliters.

Another aspect of this invention describes an elongated container containing aliquots or droplets of a biological sample(s), such as sperm-CPM or cell-CPM. According to the methods described herein, a biological sample(s) together with an elongated container may further comprise a cryostorage container defined as an elongated container containing two or more aliquots of biological sample that is frozen or cryopreserved. In one embodiment, the invention is an elongated container containing from 2 to 15 droplets, each droplet from about 5 microliters to about 50 microliters in volume, such as a volume of from about 5 microliters to about 40 microliters, a volume of from about 5 microliters to about 30 microliters, or in particular embodiments, a volume from about 5 microliters to about 10 microliters. As an example, it is feasible to place 10 or more droplets of 5 or 10 microliter sperm/CPM samples in one 250 microliter straw or within a length of an elongated container having similar volume. According to some specific embodiments, an elongated container, such as a tube, is used to store a predetermined number of droplets or aliquots of biological sample, such as sperm-CPM or cell-CPM, that are separated from each other by a volume of separation gas. The portion or segment of such elongated container containing aliquots of biological sample may be freed from the remainder of the elongated container by being cut away from it. The elongated container, such as a straw or an elongated container segment, such as a tube segment, may be further sealed at one or both ends.

Each droplet or aliquot of sample in the elongated container is separated from its nearest neighbor(s) by a volume or gap of separation gas. For example, a volume of from about 3 microliters to about 40 microliters of separation gas may be used. Where larger volume of sample aliquots or a larger number of sample aliquots are used, each aliquot or droplet of sample may be separated from its respective nearest neighbors by a gap or volume of separation gas as little as about 3 microliters. Alternatively, each droplet or aliquot of sample may be separated from its nearest neighbor(s) by a space of separation gas measured in units of distance of from about 1 mm to about 20 mm along the length of an elongated container.

The number of droplets or aliquots placed into each elongated container, such as a tube segment or straw, depends on the dimensions of the elongated container used as well as the size, properties, and number of sample droplets or aliquots. For example, 250 microliter French straws may be used. Alternatively, 300 microliter straws manufactured by Cryobiosystems (length of 133 mm and an internal diameter of 2.25 mm) or 500 microliter straws (internal diameter of 2.25 mm) may be used. In the case of cells or tissues, a 2 ml straw may be used. Alternatively, a straw having a smaller volume may be used where appropriate, such as a 250 microliter French straw. Where droplets or aliquots of biological sample are stored in an elongated container of undetermined length, such as a tube, the number and volume of aliquots or droplets that may be placed in each tube may be more variable and less limited.

It is another objective of the invention to provide methods to efficiently store cells or tissues, which is particularly useful for cells or tissues that are available in limited quantities. This is applicable to embryonic and adult stem cells, which may be totipotent, pluripotent, multipotent or unipotent stem cells. Such cells may include human embryonic stem cells, neuronal stem cells, hematopoietic stem cells, bone marrow-derived stem cells, umbilical cord-derived stem cells, cord-blood stem cells, mesenchymal stem cells, amniotic fluid-derived stem cells, endoderm stem cells, spinal cord stem cells, cardiac stem cells or fat stem cells. The present invention is also useful for the cryopreservation of primary cells, such as cells available in limited quantities (e.g., biopsy material) or cells that must be cryopreserved at an early passage to prevent differentiation and/or changes during in vitro culturing conditions. The method described herein will be useful for storing, for example, primary hepatocytes, islet cells, bone marrow cells, umbilical cord cells, and neuronal cells.

The biological sample may be further placed in a cryoprotective media prior to storage. The cryoprotective media (CPM) is composed of (1) a cryoprotectant; (2) a free radical scavenger (such as a reducing agent or an antioxidant); and optionally (2) a membrane protectant that stabilizes or assists in stabilization of membranes of sperm. The cryoprotective media may be varied in its specific components. For example, the cryoprotectant (also referred to as a cryoprotective agent) is typically a non-penetrating cryoprotectant comprising at least one non-penetrating cryoprotectant. However, a penetrating cryoprotectant comprising at least one penetrating cryoprotectant may also be used. A combination of one or more non-penetrating cryoprotectant and one or more penetrating cryoprotectant may also be used together.

A non-penetrating cryoprotectant is a solute (agent/compound) that is incapable of moving across a cell membrane of the cell or sperm. As a component of the cell suspension, it alters the nature and/or extent of changes that occur within the cell or sperm and in the cryoprotective medium to enhance or increase the number or percentage of cells or sperm that survive cryopreservation. Non-penetrating cryoprotectants useful in the present invention may be a sugar or a nonsugar or a combination of one or more sugars and one or more nonsugars. In specific embodiments, this component of the cryoprotective media is a sugar or combination of two or more sugars. Sugars included in the cryoprotective media may be disaccharides (trehalose, melibiose, sucrose), trisaccharides (e.g., raffinose, melezitose), tetrasaccharides (e.g. stachyose), oligosaccharides (mannotriose), or polysaccharides (e.g., dextran, hydroxyl-ethyl starch (HES)) and sugar alcohols thereof (e.g., maltitol, lactitol). In a particular embodiment, the sugar included in the cryoprotective media is raffinose. Nonsugars, such as polyvinyl pyrrolidone (PVP), polyethylene oxide (PEO) or polyethyleneglycol (PEG), may be included in the cryoprotective media. For example, high molecular weight PEG may be used, such as PEG of any average molecular weight from PEG400 to PEG20000 Dalton (PEG400 to PEG20000).

Alternatively, a penetrating cryoprotectant may be used. A penetrating cryoprotectant is a solute (agent/compound) or a solvent that may move across a cell membrane, such as the membrane of the sperm. This component alters the nature and/or extent of changes that occur both within the cells or sperm and in the cryoprotective media to enhance or increase the number or percentage of cells or sperm that survive cryopreservation. In specific embodiments, this component of the cryoprotective media is one or more of the following: a sugar alcohol, such as glycerol; dimethyl sulfoxide (DMSO); an alcohol, such as ethylene glycol or propylene glycol. A combination of one or more non-penetrating cryoprotectants and one or more penetrating cryoprotectants may also be used.

The membrane protectant component of the cryoprotective media may be any of a wide variety of macromolecules that act as a buffer and diluent to stabilize or assist in stabilizing cell membranes. The membrane protectant component may be a (one or more, at least one) protein, a (one or more, at least one) non-protein or a combination of a protein or proteins and a non-protein or non-proteins. The one or more proteins may be derived from animal sources (animal proteins) and may be, for example, milk, milk derivatives or components, such as milk protein, skim milk powder, casein; egg, egg yolk, egg white, collagen, elastin, gelatin, atelocollagen, fibronectin, peptones, keratin, albumin or any combination thereof. Alternatively, the one or more proteins may be derived from plant sources (plant proteins) and may be, for example, soy protein, wheat protein, corn protein, coconut milk, alovera extract, jojoba extract, or any combination thereof. The membrane protectant may be a combination of one or more animal proteins and one or more plant proteins. The one or more proteins may be a recombinantly produced protein, such as casein, collagen, gelatin, fibronectin, albumin, lactalbumin, lactoglobulin, keratin or any combination thereof. Alternatively, the membrane protectant may be a lipid (e.g., animal lipid, plant lipid, chemically synthesized); synthetic lipids, such as phosphatidylglycerol, phosphatidic acid, 1,1′,2,2′-tetra-acyl-cardiolipin, phosphatidylcholine, phosphatidylserine, phosphatidylethanolamine, polyoxyethylene based lipids, arachidonic linoleic, linolenic, myristic, oleic, palmitic or stearic fatty acids, cholesterol, Pluronic F-68 or any combination thereof may be used (e.g., as in chemically defined lipid concentrate supplies by Invitrogen Cat. No. 11905-031 or lipid mixture 1 by Sigma, Cat. No. L0288). A further example of a membrane protectant is a polymer, polyvinylalcohol (PVA).

Any combination of the membrane protectants described herein or similar agents may be used in the cryoprotective media. For example, one or more proteins, such as one or more proteins of animal origin and/or one or more proteins of plant origin and/or one or more recombinant protein may be included in the cryoprotective media, which may also comprise additional membrane protectants, such as one or more lipids (e.g., one or more lipids of animal origin and/or one or more lipids of plant origin and/or one or more synthetic lipid, any mixture of these types of lipids) and/or PVA. The concentration of the membrane protectant may be determined empirically, using methods known to those of skill in the art. In certain embodiments, the concentration of membrane protectant is from about 1% weight/volume to about 30% weight/volume, such as 10% weight/volume. Alternatively, the concentration may be from about 1% volume/volume to about 30% volume/volume, such as 10% volume/volume. In either case, the concentration may be any concentration within these ranges.

The free radical scavenger component (also referred to as a free radical scavenger agent) of the cryoprotective media may be any scavenger which is compatible with viability and functionality of sperm and sufficiently effective (active) that it removes or inactivates free radicals (reactive oxygen species) in the combination to such an extent that the number or percentage of cells or sperm that survive cryopreservation is enhanced. A free radical scavenger component may be any compound or molecule, such as a reducing agent or an antioxidant, that has these desired characteristics. Free radical scavenger agents useful in the cryoprotective media of the present invention may be identified by methods known to those of skill in the art.

One or more free radical scavengers (e.g., one or more reducing agent(s) and/or one or more antioxidants) may be included in the cryoprotective media. In specific embodiments, the free radical scavenger may be, for example, one or more reducing agents, such as one or more of the following: monothioglycerol (MTG), beta-mercaptoethanol; dithiothreitol; Tris (2-carboxyethyl) phosphine (TCEP); dithioerythritol; thioredoxin (TRX); dithionite; 2-mercaptoethylamine; dimethyl thiourea; nordihydroguaiaretic acid (NDGA); 2,3-dimercapto-1-propanol or hydroquinone. Alternatively, the free radical scavenger may be one or more antioxidants, such as one or more of the following: an amino acid or derivative thereof (e.g., glutathione (GSH); cysteine; homocysteine; N-acetyl cysteine (NAC); methionine; N-2-mercaptopropionyl glycine; alanine; glutathionine); bilirubin; melatonin; mannitol; lipoic acid; 10,11-dihydroxyaporphine (DHA); butylated hydroxyanisole (BHA); butylated hydroxytoluene; dihydroolipoic acid; tetrahydropletsapaveroline (THP); 2-thiobarbituic acid; or taurine; and dimercaptosuccinic acid inositol. Further, the free radical scavenger may be a vitamin (such as vitamin E (tocopherol or a derivative thereof, such as water soluble forms of vitamin E; vitamin C (ascorbic acid); vitamin A, carotenoids;(Astaxanthin); vitamin B complex (including inositol) or Coenzyme Q); allopurinol, dimethyl sulfoxide; deferoxamine; an enzyme (such as catalase, glutathione peroxidase or superoxide dismutase); a steroid (such as 21-aminosteroids, methylprednisolone); or glutathionine.

The elongated container generated according to the methods of the present invention may be as initially prepared (i.e., an elongated container containing aliquots of biological sample that has not yet undergo cryopreservation) or frozen/cryopreserved. Cryopreservation methods may include those known or recently developed. Cryopreservation methods further comprise any methods recently developed for the safe preservation of mouse sperm as from all inbred mouse strains (See, e.g., U.S. Provisional Patent App. Nos. 60/812,833, 60/840,744, and 60/854,501, and U.S. Utility application Ser. No. 11/811,968, the disclosures of which are hereby incorporated by reference).

It is another objective of the invention to provide methods to produce a large cohort of animals using one elongated container containing sperm. In this method, a cryopreserved elongated container of the present invention containing droplets of sperm sample is thawed rapidly and the sperm-CPM expelled into an appropriate media, such as the IVF media (See, e.g., Cooks Vitro Fertilization (Cook Australia; Queensland, Australia; (See, e.g., Quinn, P. (1995), Enhanced results in mouse and human embryo culture using a modified human tubal fluid medium lacking glucose and phosphate. J Assist Reprod Genet, 12(2), 97-105, the disclosure of which is hereby incorporated by reference))). The sperm in appropriate media is then mixed with egg masses or oocytes and cultured in vitro to fertilize the egg. Subsequently, fertilized eggs, 2-cell, 4-cell to 16-cell stage embryos, morula, or blastocyst stage embryos are introduced into an appropriate receptive female, (e.g., a pseudopregnant female), using art recognized techniques (See, e.g., Nakagata, N. (2000a). Cryopreservation of mouse sperm. Mammalian Genome 11, 572-576; Nakagata, N. (2000b). Mouse Sperm Cryopreservation. J Mamm Ova Res 17, 1-8, the disclosures of which are hereby incorporated by reference). In some contexts, such as with large animals, the thawed product could be used for artificial insemination or in vitro fertilization (IVF) procedures as described above.

It is another objective of the invention to provide methods to produce live animals u s i n g sperm stored in one cryopreserved elongated container. In this method, a cryopreserved elongated container of the present invention containing cryopreserved sperm-CPM droplets is thawed rapidly and the sperm are introduced non-surgically into an appropriate receptive female, such as into a pseudopregnant female, using art recognized techniques.

It is another objective of the invention to provide methods to produce live animals using sperm stored in a cryopreserved elongated container for surgery-assisted artificial insemination (Al) (See, e.g., Sato, M., and Kimura, M., (2001) Intrabursal transfer of sperm (ITS): A new route for artificial insemination of mice. Theriogenology 55, 1881-1890.; Sato, M., Tanigawa, M., and Watanabe, T. (2004). Effect of time of ovulation on fertilization after intrabursal transfer of sperm (ITS): Improvement of a new method for artificial insemination in mice. Theriogenology 62, 1417-1429., the disclosures of which are hereby incorporated by reference). The sperm-CPM suspension is transferred by means of a transfer pipette directly into a space between the ovary and ovarian bursa near the infundibulum of an oviduct. Alternatively, the sperm may be inserted into the ampulla region through the oviductal wall of an appropriate receptive female, such as a pseudopregnant or superovulated female.

In certain embodiments, the method of cryogenically preserving sperm comprises: combining sperm to be cryogenically preserved with a cyroprotective media (CPM) and placing the resulting sperm-CPM combination into an elongated container suitable for cryopreservation, such that at least two droplets of the sperm-CPM are placed into the elongated container and each sperm-CPM droplet is separated from its nearest neighbor(s) by a volume or gap of separation gas. In certain embodiments, such as those in which a 250 microliter French straw is used, from two to about ten sperm-CPM droplets may be introduced into each elongated container, each separated from its respective nearest neighbors by a volume or gap of separation gas of at least 3 microliters. The volume or gap of separation gas may range from about 3 microliters to about 15 microliters. In specific embodiments, the volume or gap of separation gas may be from about 3 microliters to about 5 microliters.

In certain embodiments, the method of the present invention is a method of producing live offspring, which may be vertebrates of many types, including mammals and nonmammals, using the elongated container(s) generated according to the methods of the present invention. Such method may be used to produce live offspring, such as rodents (e.g., mouse, rat), cattlelbovine (cows, bulls), dogs, cats, pigs/swine, goats, sheep, horses, camels, rabbits, other companion mammals, birds (e.g., chicken, turkey, companion birds), fish (those produced for consumption, companion fish), and other species (including endangered species). It may also be used for procedures in humans, such as IVF procedures.

The method of producing live offspring comprises the steps described above for cryogenically preserving sperm and further comprises thawing the resulting cryopreserved sperm (thereby producing thawed sperm); introducing thawed sperm or fertilized oocytes produced using thawed sperm resulting from the method described above into an appropriate female (e.g., a psuedopregnant female, such as a pseudopregnant mouse, or a superovulated female, such as a superovulated mouse) and maintaining the female under conditions appropriate for growth and development of fertilized oocytes into live offspring (appropriate for production of live offspring), whereby live offspring is/are produced. In those embodiments in which thawed sperm are introduced into an appropriate female, they are present in an appropriate media, such as the IVF media. For example, the thawed sperm-CPM combination may be diluted into an appropriate IVF media or directly introduced into a suitable receptive female. Alternatively, the thawed sperm-CPM combination is directly used in surgically assisted artificial insemination. According to yet another alternative, the thawed sperm-CPM combination is diluted into an appropriate IVF media and mixed with egg masses or oocytes and cultured in vitro to the 2-cell stage or 4-cell to 16-cell stage embryos, morula or blastocyst stage embryos. Subsequently, fertilized eggs, 2-cell stage or 4-cell to 16-cell stage embryos, morula or blastocyst stage embryos are introduced into an appropriate receptive female.

The elongated container may be filled manually or with the assistance of a device, such as a minipump, a microaspirator, or other suction-generating machine. For example, an elongated container, such as the French straw, may be filled manually through the use of a lml syringe. One end forms an airtight seal where the elongated container and the syringe join. The open end of an elongated container, such as a straw, is placed into a biological sample, and a desired volume of biological sample is drawn into the straw. If a syringe is used, the syringe plunger is retracted to pull the biological sample into the elongated container. Alternatively, the biological sample may be pulled to a marked level corresponding to the desired volume. The elongated container is taken out of the biological sample, and a desired volume of air or gas is drawn into the elongated container to separate the individual aliquots or droplets of sample from their nearest neighbor(s). This alternating process is repeated until the desired number of sample aliquots/droplets is included in the elongated container. The elongated container may be partially or completely filled. Approximately 10 mm or more of the end(s) of the elongated container may be kept clear for cutting and/or sealing. Once complete, the elongated container may be cut and/or sealed at one or both ends and may be further cut and/or sealed into smaller lengths containing one or more aliquots of sample.

The same process may be assisted by a mechanical device, which fills an elongated container with alternating volumes of sample and separation gas. This device may be used to create an airtight seal and a finely controlled vacuum within the elongated container to draw a biological sample and separation gas into the elongated container. Such a device may be further capable of manually, mechanically, or automatically moving one end of an elongated container into and out of a biological sample so that columns of biological sample and separation gas are alternately drawn into the elongated container. Alternatively, such device may instead inject or dispense a volume of biological sample, inject a volume of separation gas into the elongated container, and optionally repeat as needed. Such process may be used to partially or completely fill an elongated container with a variable number and/or volume of biological sample(s) and separation gas. This device may move from one elongated container, such as a straw or tube segment, to the next to repeat this operation, or the device may fill multiple elongated containers simultaneously.

According to yet another aspect of the present invention, an apparatus is provided with means for loading single or multiple aliquot(s), droplet(s), or volume(s) of a biological sample into an elongated container. According to some embodiments, the apparatus raises and lowers an open distal end of an elongated container, such as a tube or straw, so that it undergoes repeated cycles of contact with and withdrawal from the sample solution. Contact and withdrawal of open distal end of elongated container with the sample solution may also be achieved by raising and lowering the sample solution. The elongated container is under controlled suction pressure from a suction system, such that an alternating pattern of sample and separation gas is drawn into the elongated container as it oscillates between contact with and withdrawal from the sample, thus dividing and separating each aliquot or droplet of sample that is drawn into the elongated container by an interposed volume of separation gas drawn from the atmosphere surrounding the biological sample.

The elongated container itself directly contacts the sample solution. Using a suction system connected to an open proximal end of the elongated container, a sample may be drawn directly into the elongated container at the open distal end of the elongated container without first passing the sample through any additional tubing or surfaces that may be present within other devices.

According to some embodiments, the elongated container itself may be eventually cut, sealed, and used for storage and/or freezing of the sample. In some embodiments, the elongated container is a tubular, cylindrical or elliptical member of undetermined length. According to these embodiments, the proximal end of the tube is connected to a suction system, and the distal end of the tube is placed near the sample. A drive system of the apparatus raises and lowers the distal end of the tube such that the distal end of the tube undergoes repeated cycles of contact with and withdrawal from the sample. Since the tube is under suction pressure, an alternating pattern of sample from a vessel and separation gas from the atmosphere surrounding the tube is drawn into the tube, thus dividing and separating each aliquot or droplet of sample that is drawn into the tube by an interposed volume of separation gas. Once a desired number of volumes of sample are drawn into the tube, the tube may be cut, and one or both of the distal and proximal open ends of the tube may be sealed.

According to an alternative set of embodiments of the present invention, a drive system is used to raise and lower a vessel containing a biological sample to achieve the same basic result of repeated cycles of contact with and withdrawal from the sample by the distal end of the elongated container. According to this set of embodiments, the elongated container may be held in place, or the elongated container may be moved in unison with the sample-containing vessel by the drive system in a coordinated fashion.

The drive system and/or the suction system of the present invention may be controlled by a computer system running a software program to automate the operation of the apparatus. The computer system may control the rate or duration of each cycle by controlling the rate and timing for raising and lowering the elongated container and/or sample-containing vessel and thus the respective volumes of sample and separation gas that are drawn into the elongated container. The computer system may also control the suction system to control the amount of pressure and thus the rate of sample or separation gas flow into the elongated container during each cycle as a function of time. Thus, the computer system may allow for accurate and precise control of the amounts of sample and separation gas that are loaded into the elongated container for each cycle by controlling the drive system and/or suction system. The desired aliquot size and operation of the apparatus may thus be programmed to perform the loading procedure appropriately depending on the specific application and sample source.

The suction pressure may vary over time, or it may be continuous or constant. Further, the amount of suction may vary with each cycle of immersion into and withdrawal from the sample whereby the amount of sample or separation gas that is drawn into the tube varies over time. Where the suction system includes a syringe, for example, the computer system and associated software may control movement of the plunger of the syringe by action of a clamping or other mechanical device and a motor that are incorporated into the suction system. Where the system includes a pump, for example, the computer system and associated software may simply control the timing and amount of power supplied.

When the drive system controls the raising and lowering of the biological sample, the drive system and the suction system may be controlled by a computer system running a software program to automate the operation of the apparatus. The computer system may further control the rate or duration of each cycle by controlling the rate and timing for raising and lowering the biological sample and thus the respective volumes of sample and separation gas that are drawn into the elongated container as a function of time. The computer system may also control the suction system to control the amount of pressure and thus the rate of sample or separation gas flow into the elongated container during each cycle. The computer system may allow for accurate and precise control of the amounts of sample that are loaded into the elongated container for each aliquot. The aliquot size and operation of the apparatus may thus be programmed to perform the loading procedure appropriately depending on the specific application and sample source. By increasing the accuracy and precision of sample aliquots and separation gas volumes drawn into the elongated container, more sample aliquots may be drawn into a given length of each elongated container with less risk for error and intermixing of the aliquots. Thus, the amount and volume of sample and separation gas may vary with each cycle depending at least in part on the length of time the distal open end of the elongated container is immersed into or withdrawn from the sample.

Both of these features are particularly suitable for uses involving smaller sample sizes from perhaps smaller animal sources or where there is a limited amount of material available for cryopreservation. The computer system may be directly connected to the drive system and/or the suction system by electrical wiring, or alternatively the computer may communicate with the drive system and/or suction means wirelessly or through a computer network. Furthermore, by increasing the accuracy and precision of sample aliquots and/or separation gas volumes drawn into the elongated container through computer control of the drive system and/or suction system, it is also possible for a greater number of sample aliquots to be drawn into a given length of each elongated container with less risk for error and intermixing of the aliquots.

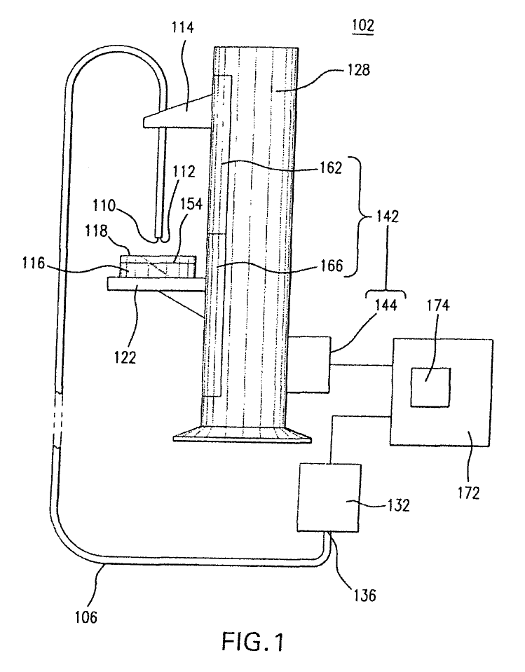

FIG. 1 illustrates various features of an apparatus 102 according to one embodiment of the present invention. An elongated container or tube 106 of undetermined length is held in place at a distal open end 110 by a tube holder 114 at a position above upper surface 154 of a biological sample 116 of interest contained in a vessel 118 such that the opening 112 at distal open end 110 of elongated container or tube 106 is pointed downward toward sample 116. Vessel 118 is held in place by a vessel support 122 relative to the position of tube holder 114 and distal open end 110 of elongated container or tube 106. Vessel support 122 may be any object or device capable of holding vessel 118 containing sample 116. For example, according to some embodiments, vessel support 122 provides a flat surface for holding a sample vessel 118. Vessel support 122 and tube holder 114 may be commonly supported by a single structure or stem 128. A suction system 132 for causing suction pressure in elongated container or tube 106 is also provided. Suction system 132 is attached to a proximal open end 136 of elongated container or tube 106.

Apparatus 102 further comprises a drive system 142 for causing movement of either (i) tube holder 114 in combination with distal open end 110 of elongated container or tube 106, or (ii) vessel support 122 in combination with vessel 118 containing sample 116. Alternatively, drive system 142 may possibly cause movement of both jointly in a coordinated fashion. Drive system 142 may include a motor 144 that powers the operation and movement of drive system 142. Where drive system 142 causes the combined movement of tube holder 114 and distal open end 110 of elongated container or tube 106 in relation to upper surface 154 of sample 116, drive system 142 includes a holder drive system 162 for mechanically raising and lowering tube holder 114 and distal open end 110 of elongated container or tube 106 in relation to vessel support 122 and vessel 118.

Alternatively, where drive system 142 causes the combined movement of vessel support 122 and vessel 118 containing sample 116, drive system 142 includes a vessel support drive system 166 for raising and lowering vessel support 122 and vessel 118 in relation to tube holder 114 and distal open end 110 of elongated container or tube 106.

Although it is presented in FIG. 1 that distal end 110 of elongated container or tube 106 is positioned directly above sample 116 at an orientation of approximately ninety degrees from upper surface 154 of sample 116, it is to be understood that drive system 142 may cause movement of (i) distal end 110 of elongated container or tube 106 via holder drive system 162, or (ii) vessel 118 and vessel support 122 via vessel support drive system 166, or both, at any angle as long as distal end 110 of elongated container or tube 106 is capable of moving toward and away from sample 116 and contacting sample 116 without being hindered by side walls of vessel 118.

In any case, the operation of the drive system 142 and/or suction system 132 may be further controlled by a computer system 172 under the direction of controlling and monitoring software 174.

According to the present invention, drive system 142 may include some combination of gearing and/or other mechanical mechanisms. The drive system may comprise, for example, a rack and pinion gearing mechanism for converting rotational motion into linear motion. Although drive system 142 may be operated by hand, drive system 142 may be automated and directly controlled by motor 144 as shown in FIG. 1. For example, the rotational force generated by motor 144 may be delivered to a gearing mechanism of drive system 142. Alternatively, drive system 142 may be operated manually. It should be appreciated that drive system 142 may cause movement of either (i) tube holder 114 and open distal end 110 of elongated container or tube 106 via holder drive system 162 in relation to upper surface 154 of sample 116, or (ii) vessel support 122 and vessel 118 containing sample 116 via vessel support drive system 166 in relation to open distal end 110 of elongated container or tube 106, or both. Further, motion generated by motor 144 may be used to impart motion to either holder drive system 162 or vessel support drive system 166, or both.

Elongated container or tube 106 of the present invention may be made of any flexible material having a diameter that is capable of drawing in and holding a sample solution or suspension under suction pressure and that is capable of being effectively cut and sealed such that the sealed elongated container, such as a straw or tube segment, may be stored or frozen. As described above, elongated container or tube 106 may be made from a clear polymeric material that is suitable for use in cryopreservation.

Vessel 118 according to the present invention may be any type capable of holding a biological sample. According to one embodiment, vessel 118 may be a Petri dish. However, according to other embodiments, vessel 118 could include other types of vessels known and used in the art, such as Falcon tubes, Eppendorf tubes, Nunc tubes, etc.

Although shown as a platform in FIG. 1, vessel support 122 of the present invention may be any type of support depending on the type of sample vessel 118 used. For example, vessel support 122 may be any type of ring, clamp, etc. that is capable of securely holding the sample vessel 118.

Although the embodiment in FIG. 1 shows that vessel support 122 and elongated container or tube 106 and tube holder 114 are commonly supported by a single structure, in other embodiments, vessel support 122 and elongated container or tube 106 and tube holder 114 may be separately supported (not shown).

Suction system 132 of the present invention may be any device capable of generating a suction pressure or a lower or negative pressure in a tube (at least transiently) relative to the surrounding environment that is sufficient to draw a sample into an elongated container, such as a tube or straw. For example, suction system 132 may include any syringe, pump, etc. that are known in the art and are capable of generating suction pressure in elongated container or tube 106.

By coordinating the movement or position of sample 116 and/or distal open end 110 of elongated container or tube 106, apparatus 102 is able to (i) immerse distal open end 110 of elongated container or tube 106 in sample 116 for a contacting period of time during which a volume, bolus, or aliquot of sample is drawn into the elongated container, and (ii) withdraw distal open end 110 of elongated container or tube 106 for a withdrawn period of time during which a volume, pocket, or gap of separation gas is drawn into the elongated container. By repeating these steps, the apparatus is able to generate an alternating pattern in the elongated container consisting of a volume of sample, followed by a volume of separation gas, followed by a volume of sample, followed by a volume of separation gas, and so on. According to some examples shown in U.S. Provisional Application No. 60/926,521, the disclosure of which is hereby incorporated by reference, storing optimal volumes of sperm in a straw such that each aliquot of sperm sample is separated from its nearest neighbor(s) by an air gap results in significantly more live born animals after recovery per volume than with single, larger volumes of sperm sample stored in each straw.

The apparatus of the present invention may further include a system for cutting off and sealing elongated container or tube 106 to generate a segment of elongated container or tube 106 containing the sample aliquots (not shown). The segment of the elongated container or tube 106 may be sealed at or near one or both of distal and proximal open ends of the segment so that the elongated container or tube segment may be frozen and/or stored. Each segment may correspond roughly to the length of the elongated container or tube 106 containing at least one of the aliquots of the sample. According to some embodiments, the tube may be heat sealed or may be sealed by a combination of heat and pressure. After cutting and/or sealing individual segments of elongated container or tube 106, each segment of elongated container or tube may also be labeled for identification purposes.

Once generated, each portion of elongated container or tube 106, such as a tube segment, may be optionally stored in cassettes or batches along with other portions of elongated container or tube 106 for freezing and/or storage. Straws of the present invention containing volumes biological samples separated by volumes of separation gas may be stored with portions of the elongated container or tube 106.

The apparatus of the present invention allows a newly generated open distal end 110 of elongated container or tube 106 to be fed into the holder 114 for the next round or application(s) and.or sample(s) once the used portion of an elongated container or tube 106 is cut away and removed from the remainder of the elongated container or tube 106.

Where elongated container or tube 106 of apparatus 102 has a unibody construction of undetermined length, such as a tube, as in the embodiment shown in FIG. 1, the apparatus may be easily reused by simply feeding a newly generated distal open end 110 of the elongated container or tube 106 (generated by cutting and removing a segment of the elongated container or tube 106 containing sample away from the remainder of the elongated container or tube 106) into tube holder 114. Since this newly generated distal end 110 of elongated container or tube 106 remains attached by its proximal open end 136 to suction system 132, apparatus 102 is immediately ready for reuse. Therefore, an unlimited number of tube segments containing a desired number of sample 116 aliquots may be generated for each sample or set of samples 116 with minimal effort expended in sealing and cutting elongated container or tube 106 and feeding a new distal end of elongated container or tube 106 into tube holder 114.

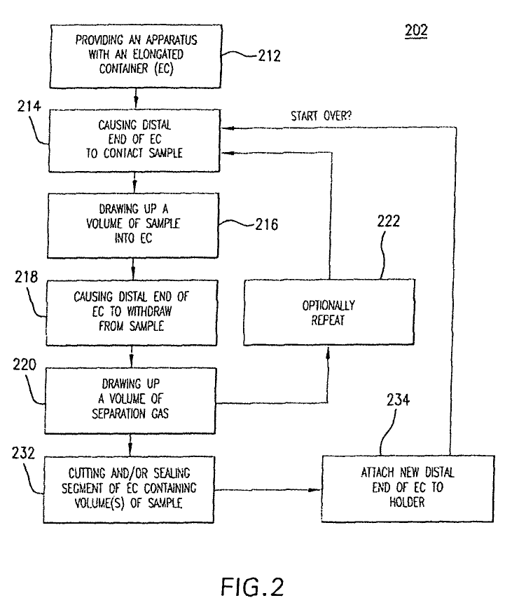

The steps of process 202 in FIG. 2 of the present invention exemplify a method of the present invention, such as a method of using the apparatus shown in FIG. 1. The steps in FIG. 2 are as follows: At step 212, distal open end 110 of elongated container or tube 106, connected at its proximal open end 136 to suction system 132 and held in place by tube holder 114 near the distal open end 110 is provided. At step 214, drive system 142 causes distal open end 110 of elongated container or tube 106 and sample 116 to move toward each other, such that distal open end 110 of elongated container or tube 106 contacts sample 116 for a period of time. This period of time may be pre-determined or selected at or near the time of contact. At step 216, a sample aliquot of desired volume is then drawn into elongated container or tube 106 due to suction pressure from suction system 132. At step 218, drive system 142 causes distal open end 110 of elongated container or tube 106 to withdraw from sample 116 for a period of time. This period of time may be pre-determined or selected at or near the time of separation. At step 220, a desired amount of separation gas is then drawn into elongated container or tube 106. At this point, the sub-process of steps 214, 216, 218 and 220 are optionally repeated as many times as desired as indicated by arrow 222. The number of cycles may be pre-determined or selected at or near the time of operation of apparatus 102.

Once the desired number of sample aliquots (separated by pockets of separation gas) has been drawn into elongated container or tube 106, at step 232, elongated container or tube 106 may be optionally cut and/or sealed to release the segment(s) of elongated container or tube 106 containing the desired sample aliquots for freezing and/or storage. The method of sealing may comprise any mechanism capable of forming a barrier between the sample aliquot(s) and the outside environment of the elongated container. For example, the segment of elongated container may be heat sealed or plugged. Each sealed segment containing sample aliquots may be optionally placed in batches or cassettes for freezing and/or storage together with other sealed elongated container segments or straws. Each sealed segment may be further labeled for identification purposes.

To repeat the above method for a new sample, round, and/or application, at step 234, the newly generated distal open end 110 of elongated container or tube 106 is fed into tube holder 114 and the sub-process of steps 214, 216, 218 and 220 may be performed again and optionally repeated as many times as desired according to arrow 222. It is to be understood that the process illustrated in FIG. 2 may be automatically controlled by a computer and its associated software.

One important advantage of the present invention as described above and illustrated in FIGS. 1 and 2 is that distal open end 110 of elongated container or tube 106 itself (that will eventually be cut and sealed for freezing and/or storage) directly contacts the sample. Therefore, after each use, the newly generated distal open end 110 of elongated container or tube 106 that remains connected to suction system 132 after cutting away the previously used elongated container segment is simply pulled into the holder in preparation for use with the next application and/or sample. There is no internal tubing or surfaces within the device or apparatus that would come in contact with sample before loading the elongated container or tube that would have to be cleaned and/or removed between applications, which may be time and labor intensive. As a result, there is little or no risk of cross-contamination between different samples and/or applications because the newly generated distal open end 110 of elongated container or tube 106 will not have come in contact with sample 116 during the previous use or application.

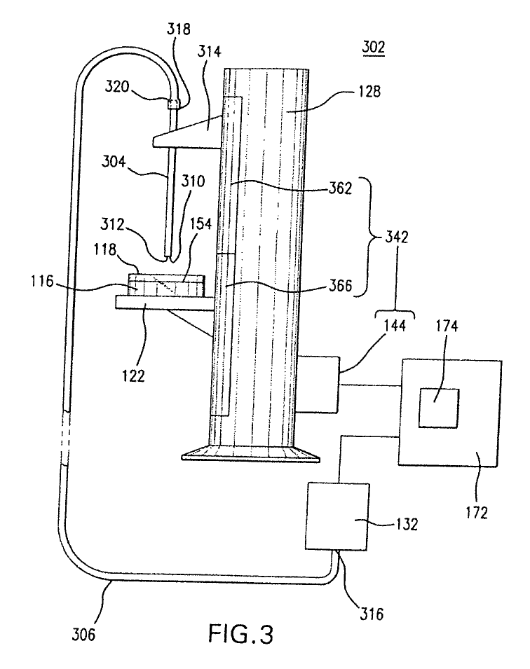

Another advantage of the present invention is that an elongated container of undetermined length (and potentially very long length) may be used, such that little time or effort is expended between different applications, samples, and/or uses since the newly generated distal open end of the elongated container is simply pulled into the holder. Similar advantages are provided for embodiments of the present invention wherein a straw is connected to the distal end of an elongated container or tube with the elongated container or tubing is further connected to the suction system as described below and shown in FIG. 3.

According to some embodiments, the suction system of the apparatus of the present invention may be additionally connected to a pre-cut straw via an elongated container or tube. According to these embodiments, the elongated container or tube is connected to the suction system at its proximal end as before. However, instead of the distal end of the elongated container or tube contacting the sample, the distal end of the elongated container or tube is connected to a pre-cut straw at one end, which in turn directly contacts the sample at its opposite end. In basically all other aspects, such apparatus may be described as provided above.

A drive system of the apparatus raises and lowers the straw to allow at least one end of the straw to undergo repeated cycles of contact and withdrawal from the sample. Since the straw is under suction pressure via the suction system and elongated container or tube, an alternating pattern of sample and separation gas from the atmosphere surrounding the straw is drawn into the straw as it oscillates between contact with and withdrawal from the sample, thus dividing and separating each aliquot or droplet of sample that is drawn into the straw by an interposed volume of separation gas. Once a desired number of volumes of sample are drawn into the straw, the straw may be detached from the distal end of the elongated container or tubing. The straw may be further cut and/or sealed at one or both ends. As described before, contact and withdrawal of the straw from the sample may alternatively be achieved by having a drive system that causes movement of the sample-containing vessel in relation to at least one end of the straw.

FIG. 3 illustrates various features of an apparatus 302 according to one embodiment of the present invention. A proximal end 320 of straw 304 is attached to distal end 318 of an elongated container or tube 306. As a means of attachment, straw 304 is shown as being inserted snugly into the lumen of elongated container or tube 306 and held by friction. However, it is to be understood that other arrangements may be envisioned, including, for example, having straw 304 fit snugly around the exterior of the elongated container or tube 306 (not shown). Straw 304 is held in place by a straw holder 314 at a position above upper surface 154 of a biological sample 116 contained in a vessel 118 such that the opening 312 at distal open end 310 of straw 304 is pointed downward toward sample 116. Vessel 118 is held in place by a vessel support 122 relative to the position of straw holder 314 and distal open end 310 of straw 304. Vessel support 122 may be any object or device capable of holding vessel 118 containing sample 116. As an example, vessel support 122 may provide a flat surface for holding a sample vessel 118 as shown. Vessel support 122 and straw holder 314 may be commonly supported by a single structure or stem 128 as shown. A suction system 132 is provided for causing suction pressure in straw 304 via the elongated container or tube 306. Suction system 132 is attached to proximal open end 316 of elongated container or tube 306 which is connected in turn to proximal end 320 of straw 304 at the distal end 318 of the elongated container or tube 306.

Apparatus 302 also includes a drive system 342 for causing movement of either (i) straw holder 314 in combination with distal open end 310 of straw 304 or (ii) vessel support 122 in combination with vessel 118 containing sample 116, or possibly both combinations jointly in a coordinated fashion. Drive system 342 may include a motor 144 used to power the operation and movement of drive system 342 as shown. Where drive system 342 causes movement of straw holder 314 and distal open end 310 of straw 304 toward and away from upper surface 154 of sample 116, drive system 342 further includes a holder drive system 362 for mechanically raising and lowering straw holder 314 and distal open end 310 of straw 304 in relation to vessel support 122 and vessel 118.

Alternatively, where drive system 342 causes a combined movement of vessel support 122 and sample-containing vessel 118 toward and away from open distal end 310 of straw 304, drive system 342 includes a vessel support drive system 366 for raising and lowering vessel support 122 and vessel 118 in relation to straw holder 314 and distal open end 310 of straw 304.

Although it is presented in FIG. 3 that distal end 310 of straw 304 is positioned directly above sample 116 at an orientation of approximately ninety degrees from upper surface 154 of sample 116, it is to be understood that drive system 342 may cause movement of (i) distal end 310 of straw 304 via holder drive system 362, or (ii) vessel 118 and vessel support 122 via vessel support drive system 366, or both, at any angle as long as distal end 310 of straw 304 is capable of moving toward and away from sample 116 and contacting sample 116 without being hindered by side walls of vessel 118.

In any case, the operation of drive system 342 and/or suction system 132 may be further controlled by a computer system 172 under the direction of controlling and monitoring software 174.

According to the present invention, drive system 342 may include some combination of gearing and/or other mechanical mechanisms. Drive system 342 may comprise, for example, a rack and pinion gearing mechanism for converting rotational motion into linear motion. Although drive system 342 may be operated by hand, drive system 342 may be automated and directly controlled by motor 144 as shown in FIG. 3. For example, the rotational force generated by motor 144 may be delivered to a gearing mechanism of drive system 342. Alternatively, drive system 342 may be operated manually.

It should be appreciated that drive system 342 may cause movement of either (i) straw holder 314 and open distal end 310 of straw 304 via holder drive system 362 in relation to upper surface 154 of sample 116, or (ii) vessel support 122 and vessel 118 containing sample 116 via vessel support drive system 366 in relation to open distal end 310 of straw 304, or both. Further, motion generated by motor 144 may be used to impart motion to either holder drive system 362 or vessel support drive system 366, or both.

Vessel 118 according to the present invention may be any type capable of holding a biological sample. According to one embodiment, vessel 118 may be a Petri dish. However, according to other embodiments, vessel 118 could include other types of vessels known and used in the art, such as Falcon tubes, Eppendorf tubes, Nunc tubes, etc.

Although shown as a platform in FIG. 3, vessel support 122 of the present invention may be any type of support depending on the type of sample vessel 118 used. For example, vessel support 122 may be any type of ring, clamp, etc. that is capable of securely holding the sample vessel 118.

Although the embodiment in FIG. 3 shows that vessel support 122 and straw 304 and straw holder 314 are commonly supported by a single structure, in other embodiments, vessel support 122 and straw 304 and straw holder 314 may be separately supported (not shown).

Suction system 132 of the present invention may be any device capable of generating a suction pressure or a lower or negative pressure in a tube (at least transiently) relative to the surrounding environment that is sufficient to draw a sample into an elongated container, such as a tube or straw. For example, suction system 132 may include any syringe, pump, etc. that are known in the art and are capable of generating suction pressure in elongated container or tube 306 as well as straw 304.

The apparatus of the present invention may further include a system for sealing each straw 304 containing the sample aliquots (not shown). The straw 304 may be sealed at or near one or both distal and proximal open ends of the segment so that straw 304 may be frozen and/or stored. According to some embodiments, the tube may be heat sealed or may be sealed by a combination of heat and pressure. Alternatively, the straw may be plugged at one or both ends. Once generated, each straw 304 may be optionally stored in cassettes or batches along with other straws or tube portions/segments for freezing and/or storage.

With the major exception of using a straw, the embodiment in FIG. 3 is largely the same as the embodiment shown in FIG. 1. In a fashion similar to the cycle described for the embodiment of FIG. 1, the cycle of contact and withdrawal of the straw with a biological sample may be repeated once or many times to generate multiple sample aliquots in the straw interposed by volumes of separation gas to separate each aliquot of sample in the straw. In this way, an unlimited number of straws containing the desired number of sample aliquots may be generated for each sample or set of samples with minimal effort expended in exchanging straws between various rounds, samples, and/or applications with each straw accommodating up to ten or more sample aliquots. Again, by automating the apparatus through the use of a computer system and software, the accuracy and precision of sample aliquot and separation gas volumes in each straw is improved. Thus, a greater number of sample aliquots (e.g., ten or more) may potentially be accommodated by each straw.

The present invention further comprises a method for drawing an alternating pattern of sample and separation gas into the straw using an apparatus much like the embodiment shown in FIG. 3. Again, one of the key aspects of this method is the ability to control the timing and volumes of alternating sample aliquots and separation gas in a straw through coordinated control of the suction system and the drive system. As a result, an accurate and precise volume of sample and separation gas in possibly varying amount may be drawn into the straw during each cycle depending on the sample type, size, and source as well as their intended and eventual use or application following storage and/or freezing.

As described above, a further advantage of the method of the present invention is that the distal open end of the straw directly contacts the sample solution. The cycle of contact and withdrawal of distal open end of the straw with the sample solution may be repeated one or more times to generate multiple sample aliquots in the straw interspersed by volumes of separation gas to separate each aliquot of sample in the straw.

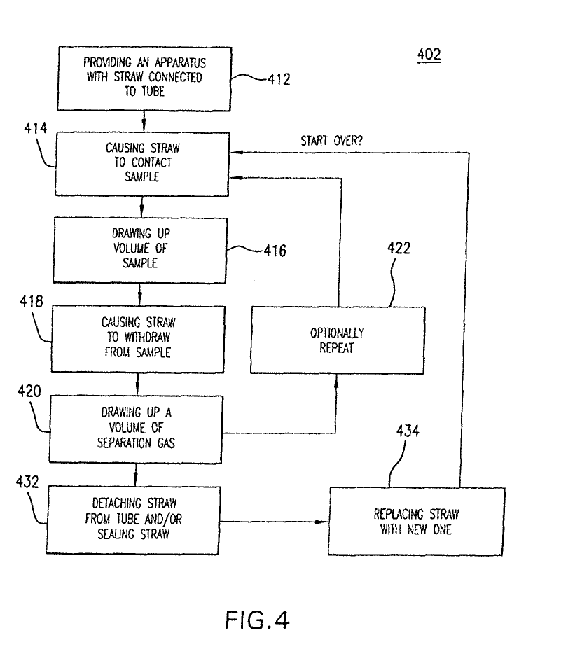

The steps of a process 402 of the present invention using an apparatus much like the embodiment shown in FIG. 3 is provided in FIG. 4. The steps in FIG. 4 are as follows: At step 412, a straw 304 connected to a distal open end 310 of an elongated container or tube 306 under suction pressure via a suction system 132 is provided and held in place by a straw holder 314 relative to the position of the sample. At step 414, drive system 342 causes the distal open end 310 of straw 304 and sample 116 to move toward each other such that distal open end 310 of straw 304 contacts the sample for a contacting period of time. This period of time may be pre-determined or selected at or near the time of contact. At step 416, a sample aliquot of desired volume is drawn into straw 304 due to the suction pressure generated by the suction system 132 via the elongated container or tube 306. At step 418, drive system 342 causes the distal open end 310 of straw 304 to move away from each other such that straw 304 withdraws from sample 116 for a withdrawn period of time. This period of time may be pre-determined or selected at or near the time of separation. At step 420, a desired amount of separation gas is then drawn into straw 304. At this point, the sub-process of steps 414, 416, 418 and 420 may be optionally repeated as many times as desired as indicated by arrow 422.

Once the desired number of sample aliquots separated by separation gas pockets has been drawn into straw 304, at step 432, straw 304 containing the desired sample aliquots is detached from the elongated container or tube 306. To repeat the above method for a new sample, round, and/or application, at step 434, a new straw 304 is attached to distal open end 310 of the elongated container or tube 306 and placed into the straw holder 314 so that the sub-process of steps 414, 416, 418 and 420 may be performed again and optionally repeated 422. It is to be understood that the process illustrated FIG. 4 may be automatically controlled by a computer and associated software.

At step 432, a detached straw 304 may be optionally cut and/or sealed for freezing and/or storage. The method of sealing may comprise any mechanism capable of forming a barrier between the sample aliquot(s) and the outside environment of the tube. For example, the straw may be heat sealed or plugged. Each sealed straw containing sample aliquots may be optionally placed in batches or cassettes for freezing and/or storage together with other sealed tube segments and/or straws.

The method of the present invention allows for a proximal end 320 of a new straw 304 to be attached to the distal end 318 of elongated container or tube 306 and straw holder 314 for the next round or application(s) and/or sample(s) once a straw 304 previously used has been removed from the elongated container or tube 306.

The biological samples of the present invention may be any type of biological is capable of being drawn into the elongated container. According to some embodiments, the sample is a suspension of sperm. Once the desired number of sample aliquots of appropriate volume is loaded into the elongated container, the elongated container may be cut and sealed at least on either or both ends of the length of tube or straw containing the sample aliquots so that the sealed length of tube or straw may be frozen and/or stored for later use.

Although both FIG. 1 and FIG. 3 depict an apparatus with a single elongated container contacting sample, it is to be understood that apparatus and method of present invention may further include multiple elongated containers, such as straws and/or tubes, aligned in parallel to contact and withdraw from sample together. Such elongated containers may further be attached to suction system via a multiport manifold.

### EXAMPLES

### Example 1

### Cryopreservation and Recovery of Sperm from C57BL/6J Mouse Inbred Strain in Different Volumes

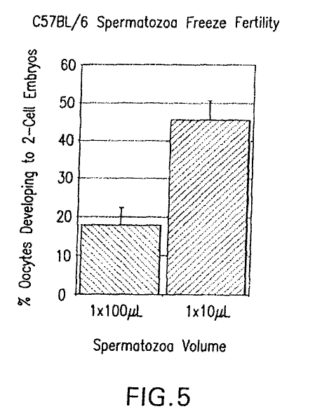

By way of example, the epididymides and vas deferentia are extracted from a C57BL/6J male mouse and placed into the cryoprotective media described herein. For example, they are extracted and placed into an incubated 1 mL collection droplet of CryoProtective Medium (CPM), which comprises, for example, raffinose (18%w/v), skim milk (3% [w/v]) and monothioglycerol (MTG) as described, for example, in U.S. Provisional App. Nos. 60/812,833; 60/840,744; and 60/854,501, and U.S. Utility application Ser. No. 11/811,968, the disclosures of which are hereby incorporated by reference. If more than one male of the same strain is collected, the sperm can be combined, and the collection droplet volume is adjusted to one male/mL. For example, 2 mL of CPM are used if two males are used. Incisions are made in the tissues, allowing the sperm to swim out into the CPM; this is allowed for at least 1 min but for less than 60 min. The residual tissues are then removed from the collection droplets. In one case, 10 microliters of the sperm sample are loaded into as many as two hundred 250-microliter French straws (IMV; Maple Grove, Minn.). In the other case one hundred micro liters of sperm sample are loaded into the 250-microliter straws. The straws are sealed with an instantaneous heat sealer. The straws are loaded into cassettes and exposed to liquid nitrogen vapor for at least 10 min before being plunged into the liquid nitrogen. Subsequent to storage in liquid nitrogen, 3 samples from each treatment are thawed in a 37° C. water bath for 10 to 30 sec, and then each 10 microliter aliquot is placed directly into its own 500 microliter in vitro fertilization (IVF) droplets of Cooks Mouse Vitro Fertilization (Cook Australia; Queensland, Australia). After incubation from 10 min to 90 min, 2 to 6 cumulus intact oocyte clutches from superovulated C57BL/6J females are added to the in vitro fertilization droplets. IVF is carried out using methods known to those of skill in the art, such as those described above. The sperm and oocytes are incubated together for 4 hrs before the presumptive zygotes are removed from the IVF droplets and placed in a culture for overnight incubation. Approximately 18 hours later, the percentage of oocytes developing into 2-cell embryos is determined (#2-cell embryos/# total oocytes); see Table 1 and FIG. 5. The percentage of 2-cell embryos from 1×10 microliter volumes of recovered sperm was about 45%, whereas the percentage of 2-cell embryos from 1×100 microliter volumes was only about 18%.

TABLE 1
C57BL/6J Sperm Fertility depending on Freeze Volume.

| | | Total | | | | |
|---|---|---|---|---|---|---|
| | | One-cell | 2-cell | | | |
| | | Stage | Embryos | Dead | | |
| Sperm | | before | after | after | Fragmented | % 2-cell |
| Volume | | IVF | IVF | IVF | after IVF | Embryos |
| 1 × 100 | uL | 89 | 40 | 0 | 2 | 45 |
| 1 × 100 | uL | 47 | 3 | 0 | 0 | 6 |
| 1 × 100 | uL | 52 | 4 | 0 | 5 | 8 |
| 1 × 100 | uL | 72 | 8 | 0 | 1 | 11 |
| 1 × 100 | uL | 63 | 16 | 0 | 0 | 25 |
| 1 × 100 | uL | 56 | 7 | 0 | 5 | 13 |
| 1 × 100 | uL | 62 | 13 | 0 | 0 | 21 |
| 1 × 100 | uL | 46 | 2 | 0 | 0 | 4 |
| 1 × 100 | uL | 44 | 10 | 0 | 4 | 23 |
| 1 × 10 | uL | 41 | 19 | 0 | 0 | 46 |
| 1 × 10 | uL | 53 | 36 | 0 | 2 | 68 |
| 1 × 10 | uL | 66 | 27 | 0 | 1 | 41 |
| 1 × 10 | uL | 38 | 19 | 0 | 2 | 50 |
| 1 × 10 | uL | 91 | 42 | 0 | 0 | 46 |
| 1 × 10 | uL | 67 | 20 | 0 | 2 | 30 |
| 1 × 10 | uL | 63 | 33 | 0 | 2 | 52 |
| 1 × 10 | uL | 71 | 35 | 0 | 0 | 49 |
| 1 × 10 | uL | 68 | 11 | 0 | 5 | 16 |

### Example 2

### Cryopreservation and Recovery of Sperm from C57BL/6J Mouse Inbred Strain in Different Configurations

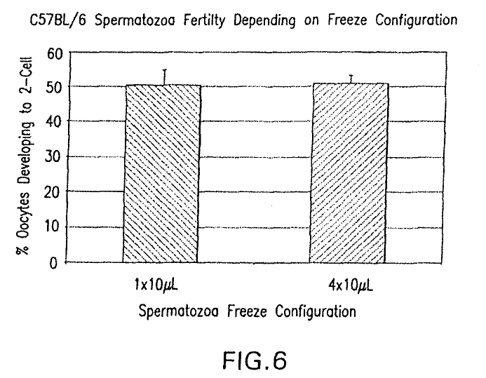

C57BL/6J sperm were isolated as described in Example 1. Ten micro liters of the sperm sample were loaded into as many as two hundred 250 microliter French straws (IMV; Maple Grove, Minn.). Alternatively, ten micro liters of sperm sample is filled, followed by an air gap (about 5-10 microliter), followed by sperm solution and so on, until four sperm samples are placed in one straw. The straws are loaded into cassettes and exposed to liquid nitrogen vapor for at least 10 min before being plunged into the liquid nitrogen. Subsequent to storage in liquid nitrogen, samples from each treatment are thawed in a 37° C. water bath for 30 sec, and then each 10 microliter aliquot is placed directly into its own 500 microliter in vitro fertilization (IVF) droplets of Cooks Mouse Vitro Fertilization (Cook Australia; Queensland, Australia). After an hour of incubation, 4 cumulus intact oocyte clutches from superovulated C57BL/6J females are added to the in vitro fertilization droplets. IVF is carried out using methods known to those of skill in the art, such as those described above. The sperm and oocytes are incubated together for 4 hrs before the presumptive zygotes are removed from the IVF droplets and placed in culture droplets for overnight incubation. Approximately 18 hours later, the percentage of oocytes developing into 2-cell embryos is determined (#2-cell embryos/# total oocytes); see Table 2 and FIG. 6. Whether sperm were recovered from a 1×10 microliter volume or 4×10 microliter volumes, the fertilization efficiency (i.e., % 2-cell stage) was roughly the same (approximately 51%).

TABLE 2
C57BL/6J Sperm Fertility depending on Freeze Configuration.

| | 2-cell stage | One cell | |
|---|---|---|---|
| | Embryos | stage after | % 2- |
| Sperm Volume | after IVF | IVF | cell Embryos |
| 1 × 10 uL | 25 | 21 | 54 |
| 1 × 10 uL | 35 | 23 | 60 |
| 1 × 10 uL | 20 | 23 | 47 |
| 1 × 10 uL | 56 | 23 | 71 |
| 1 × 10 uL | 44 | 54 | 45 |
| 1 × 10 uL | 37 | 50 | 43 |
| 1 × 10 uL | 29 | 64 | 31 |
| 1 × 10 uL | 27 | 23 | 54 |
| 4 × 10 uL | 42 | 19 | 69 |
| 4 × 10 uL | 28 | 21 | 57 |
| 4 × 10 uL | 44 | 46 | 49 |
| 4 × 10 uL | 32 | 41 | 44 |
| 4 × 10 uL | 34 | 29 | 54 |
| 4 × 10 uL | 44 | 51 | 46 |
| 4 × 10 uL | 36 | 32 | 53 |
| 4 × 10 uL | 15 | 19 | 44 |
| 4 × 10 uL | 17 | 18 | 49 |
| 4 × 10 uL | 29 | 28 | 51 |
| 4 × 10 uL | 26 | 41 | 39 |
| 4 × 10 uL | 28 | 21 | 57 |

### Example 3

### Fertilization Efficiency of Sperm Recovered from Different Volumes

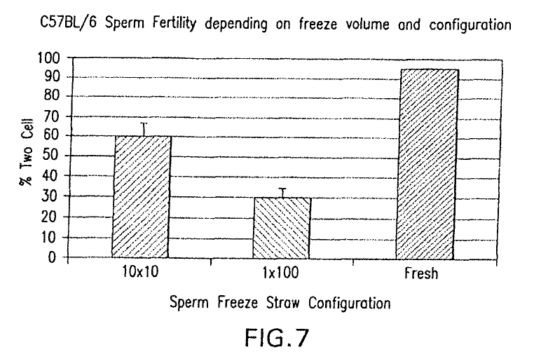

According to another set of experiments, C57BL/6J sperm were extracted as described above and frozen in straws in 1×100 microliter or 10×10 microliter configurations. Three straws from each configuration were thawed, and each straw was used to populate three IVF dishes with sperm (for a total of nine dishes per configuration). Sperm were incubated in the IVF dishes for 75 minutes before 3-4 C57BL/6J clutches were added to each IVF dish. As a control, two dishes of fresh C57BL/6J sperm were also used. Eggs were washed after 4 hours of co-incubation with the sperm and allowed to remain in culture overnight. Two-cell embryos were counted 25 hours post-IVF to measure fertilization frequency. See Table 3; FIG. 7. The average % fertilization for the 10×10 microliter arrangement was about 59.56% (SEM 7.3), whereas the average % fertilization for the 1×100 microliter volume was about 29.7% (SEM 4.7). The average % fertilization for the control group of fresh sperm was about 94.5% (SEM 0.5).

TABLE 3
C57BL/6 Sperm fertility depending on freeze volume and configuration.

| Volume Configuration | % Fertilization |
|---|---|
| or Treatment | (% 2-cell stage embryos) |
| 10 × 10 microliter | 40 |
| 10 × 10 microliter | 59 |
| 10 × 10 microliter | 100 |
| 10 × 10 microliter | 65 |
| 10 × 10 microliter | 58 |
| 10 × 10 microliter | 62 |
| 10 × 10 microliter | 66 |
| 10 × 10 microliter | 19 |
| 10 × 10 microliter | 67 |
| 1 × 100 microliter | 26 |
| 1 × 100 microliter | 37 |
| 1 × 100 microliter | 36 |
| 1 × 100 microliter | 43 |
| 1 × 100 microliter | 7 |
| 1 × 100 microliter | 5 |
| 1 × 100 microliter | 22 |
| 1 × 100 microliter | 38 |
| 1 × 100 microliter | 50 |
| 1 × 100 microliter | 33 |
| Fresh sperm control | 95 |
| Fresh sperm control | 94 |

### Example 4

### Fertilization Efficiency Across a Range of Freezing Volumes

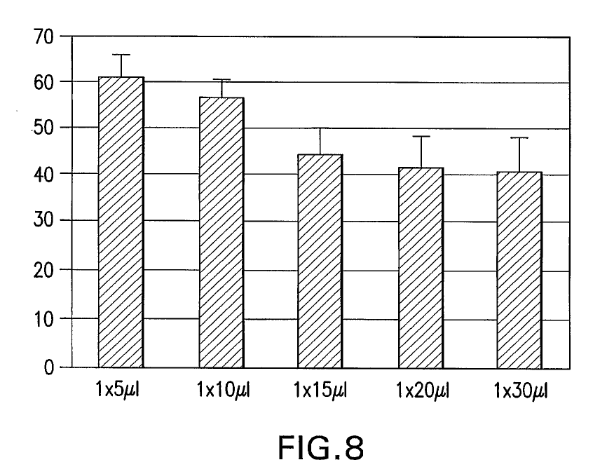

C57BL/6J sperm were isolated as described in Example 1. Five, ten, fifteen, twenty, thirty or one hundred microliters of sperm sample are loaded into as many as one hundred 250 microliter French straws (IMV; Maple Grove, Minn.). The straws are loaded into cassettes and exposed to liquid nitrogen vapor for at least 10 min before being plunged into the liquid nitrogen. Subsequent to storage in liquid nitrogen, samples from each treatment are thawed in a 37° C. water bath for 30 sec, and then each 10 microliter aliquot is placed directly into its own 500 microliter in vitro fertilization (IVF) droplets of Cooks Mouse Vitro Fert (Cook Australia; Queensland, Australia). In the case of the five microliter freeze, five microliter are used for the IVF. After an hour of incubation, 4 cumulus intact oocyte clutches from superovulated C57BL/6J females are added to the in vitro fertilization droplets. IVF is carried out using methods known to those of skill in the art, such as described above. The sperm and oocytes are incubated together for 4 hrs before the presumptive zygotes are removed from the IVF droplets and placed in a culture for overnight incubation. Approximately 18 hours later, the percentage of oocytes developing into 2-cell embryos is determined (#2-cell embryos/# total oocytes); see Table 4 and FIG. 8. Please note that the 1×30 microliter data is not shown in FIG. 8 to allow for clarity of the figure.

The average % fertilization for the 1×5 microliter volume was 60.78% (SEM 4.74), the average % fertilization for the 1×10 microliter volume was 56.89% (SEM 3.73), the average % fertilization for the 1×15 microliter volume was 45.33% (SEM 6.34), and the average % fertilization for the 1×20 microliter volume was 37.63% (SEM 6.36).

TABLE 4
Relationship between sperm volume and fertilization frequency.

| Volume | % Fertilization |
|---|---|
| Configuration in Straw | (% 2-cell stage embryos) |
| 1 × 5 microliter | 63 |
| 1 × 5 microliter | 51 |
| 1 × 5 microliter | 35 |
| 1 × 5 microliter | 68 |
| 1 × 5 microliter | 53 |
| 1 × 5 microliter | 61 |
| 1 × 5 microliter | 76 |
| 1 × 5 microliter | 83 |
| 1 × 5 microliter | 57 |
| 1 × 10 microliter | 34 |
| 1 × 10 microliter | 49 |
| 1 × 10 microliter | 62 |
| 1 × 10 microliter | 69 |
| 1 × 10 microliter | 56 |
| 1 × 10 microliter | 55 |
| 1 × 10 microliter | 55 |
| 1 × 10 microliter | 72 |
| 1 × 15 microliter | 60 |
| 1 × 15 microliter | 48 |
| 1 × 15 microliter | 25 |
| 1 × 15 microliter | 30 |
| 1 × 15 microliter | 45 |
| 1 × 15 microliter | 23 |
| 1 × 15 microliter | 34 |
| 1 × 15 microliter | 73 |
| 1 × 15 microliter | 59 |
| 1 × 20 microliter | 71 |
| 1 × 20 microliter | 18 |
| 1 × 20 microliter | 48 |
| 1 × 20 microliter | 16 |
| 1 × 20 microliter | 42 |
| 1 × 20 microliter | 29 |
| 1 × 20 microliter | 30 |
| 1 × 20 microliter | 70 |
| 1 × 20 microliter | 48 |
| 1 × 30 microliter | 15 |
| 1 × 30 microliter | 25 |
| 1 × 30 microliter | 29 |
| 1 × 30 microliter | 91 |
| 1 × 30 microliter | 37 |
| 1 × 30 microliter | 48 |
| 1 × 30 microliter | 57 |
| 1 × 30 microliter | 26 |
| 1 × 30 microliter | 41 |
| 1 × 30 microliter | 37 |

### Example 4

### Producing Live Born Mice from Multi-Droplets Straws

Sperm isolated and cryopreserved as described in Examples 1 and 2 may be thawed as described. The thawed sperm may be used for IVF as carried out using methods known to those of skill in the art, such as described above. Briefly, sperm and oocytes are incubated together for 4 hrs before the presumptive zygotes are removed from the IVF droplets and placed in culture droplets for overnight incubation. After 18 hours the number of 2-cell embryos may be determined. The 2-cell embryos may be either cultured further, up to the blastocyst stage or transferred directly to pseudopregnant female recipients. Alternatively, the thawed sperm may be used directly in surgical assisted insemination using methods described above.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

What is claimed is:

1. A method, comprising:
(i) placing at least two volumes of biological sample in an elongated container wherein each of said volumes of biological sample in said elongated container is separated from each of its nearest neighbor(s) by a volume of separation gas, and
(ii) cryogenically freezing said elongated container.

2. A method, comprising:
(i) placing at least two volumes of biological sample in an elongated container, wherein each of said volumes of biological sample in said elongated container is separated from all other of said volumes of biological sample by at least one volume of separation gas, and
(ii) cryogenically freezing said elongated container.

3. A method, comprising:
(i) placing at least two volumes of a biological sample in an elongated container, wherein all of said volumes of biological sample in said elongated container are separated from all other of said volumes of biological sample by at least one volume of separation gas, and
(ii) cryogenically freezing said elongated container.

4. The method of claim 3, wherein said separation gas comprises air.

5. The method of claim 3, wherein said biological sample contains a cryoprotective media (CPM).

6. The method of claim 5, wherein said biological sample comprises a sperm-CPM sample.

7. The method of claim 6, wherein the volume of each of said sperm-CPM samples in said elongated container is from about 5 microliters to about 60 microliters and each of said volumes of separation gas is from about 3 microliters to about 40 microliters.

8. The method of claim 7, wherein the volume of each of said sperm-CPM samples is from about 5 microliters to about 30 microliters.

9. The method of claim 8, wherein the volume of each of said sperm-CPM samples is from about 5 microliters to about 15 microliters.

10. The method of claim 6, wherein at least two of said sperm-CPM samples is separated by a volume of separation gas comprising a space of from about 1 mm to about 20 mm in distance along the long axis of said elongated container.

11. The method of claim 5, wherein said biological sample comprises a cell-CPM sample.

12. The method of claim 3, wherein said elongated container is a straw.

13. The method of claim 12, further comprising the step of: sealing said straw at one or both ends prior to said cryogenically freezing step.

14. The method of claim 3, wherein said elongated container is a tube of undetermined length.

15. The method of claim 14, further comprising the step of: cutting said tube prior to said cryogenically freezing step to produce a segment of said tube containing said volumes of biological sample.

16. The method of claim 15, further comprising the step of: sealing said segment of said tube at one or both ends.

17. An article comprising:
(i) an elongated container; and
(ii) at least two volumes of biological sample, wherein said volumes of biological sample are placed in said elongated container, wherein each of said volumes of biological sample is separated from each of its nearest neighbor(s) in said elongated container by a volume of separation gas and wherein said article is a cryostorage container.

18. An article comprising:
(i) an elongated container; and
(ii) at least two volumes of biological sample contained in said elongated container, wherein each of said volumes of biological sample is separated from all other of said volumes of biological sample in said elongated container by at least one volume of separation gas and wherein said article is a cryostorage container.

19. A article comprising:
(i) an elongated container; and
(ii) at least two volumes of biological sample contained in said elongated container, wherein all of said volumes of biological sample are separated from all other of said volumes of biological sample in said elongated container by at least one volume of separation gas and wherein said article is a cryostorage container.

20. The article of claim 19, wherein said separation gas comprises air.

21. The article of claim 19, wherein said biological sample contains a cryoprotective media (CPM).

22. The article of claim 21, wherein said biological sample comprises a sperm-CPM sample.

23. The article of claim 22, wherein the volume of each of said sperm-CPM samples is from about 5 microliters to about 60 microliters and each of said volumes of separation gas is from about 3 microliters to about 40 microliters.

24. The article of claim 23, wherein the volume of each of said sperm-CPM samples is from about 5 microliters to about 30 microliters.

25. The article of claim 24, wherein the volume of each of said sperm-CPM samples is from about 5 microliters to about 15 microliters.

26. The article of claim 22, wherein at least two of said sperm-CPM samples is separated by a volume of separation gas comprising a space of from about 1 mm to about 20 mm in distance along the long axis of said elongated container.

27. The article of claim 22, wherein said sample of sperm is from a vertebrate species.

28. The article of claim 27, wherein said sample of sperm is from a mammalian species.

29. The article of claim 28, wherein said sample of sperm comprises rodent sperm.

30. The article of claim 29, wherein said sample of sperm comprises human sperm.

31. The article of claim 19, wherein said cryostorage container is cryogenically frozen.

32. The article of claim 19, wherein said elongated container is a straw.

33. The article of claim 32, wherein said straw is further sealed at one or both ends.

34. The article of claim 19, wherein said elongated container is a tube of undetermined length.

35. The article of claim 34, wherein said tube comprises a segment of said tube containing said volumes of biological sample that is generated and released from the remainder of said tube by cutting said tube.

36. The article of claim 35, wherein said segment of said tube is further sealed at one or both ends.

37. The article of claim 23, wherein said elongated container is a 250 microliter French straw.

38. The article of claim 21, wherein said biological sample comprises a cell-CPM sample.

39. The article of claim 38, wherein said cells comprise primary cells.

40. The article of claim 39, wherein said primary cells are selected from the group consisting of hepatocytes, islet cells, endothelial cells, and nerve cells.

41. The article of claim 39, wherein said cells are stem cells.

42. The article of claim 41, wherein the stem cells are selected from the group consisting of adult stem cells, embryonic stem cells, mesenchymal stem cells, neuronal stem cells, bone marrow stem cells, umbilical cord stem cells, spermatogonial stem cells, and amniotic-fluid derived stem cells.

43. A method of producing live offspring, comprising:
(i) thawing at least one elongated container containing at least two volumes of a sperm-CPM sample to produce a thawed elongated container, each of said sperm-CPM samples in said elongated container being separated from all other of said sperm-CPM samples by at least one volume of separation gas;
(ii) introducing sperm obtained from said thawed elongated container to at least one oocyte to produce at least one fertilized oocyte; and
(iii) placing said at least one fertilized oocyte into one or more appropriate females under appropriate conditions for growth and development into live offspring.

44. A method of producing live offspring, comprising:
(i) thawing at least one elongated container containing at least two volumes of a sperm-CPM sample to produce a thawed elongated container, each of said sperm-CPM samples in said elongated container being separated from all other of said sperm-CPM samples by at least one volume of separation gas; and
(ii) introducing sperm obtained from said thawed elongated container into one or more appropriate females.

45. An apparatus, comprising:
(i) an elongated container having an open proximal end and an open distal end;
(ii) a drive system that causes movement of said distal end of said elongated container; and
(iii) a suction system that causes a suction pressure in said elongated container, wherein said suction system is connected to said proximal end of said elongated container, and wherein said drive system is able to reversibly and repeatedly cause said distal end of said elongated container to contact and withdraw from a biological sample contained in a vessel.

46. An apparatus, comprising:
(i) a vessel containing a biological sample;
(ii) a vessel support for holding said vessel;
(iii) an elongated container of undetermined length having a proximal end and a distal end;
(iv) a holder for holding said elongated container near said distal end such that said distal end is positioned above said biological sample with the opening of said distal end of said tube pointing downward toward said biological sample;
(v) a drive system that causes movement of said holder and said distal end of said elongated container toward and away from said biological sample; and
(vi) a suction system that causes a suction pressure in said elongated container, wherein said suction system is attached to said proximal end of said elongated container.

47. An apparatus, comprising:
(i) a unibody elongated container having an open distal end and an open proximal open end;
(ii) a drive system that is capable of moving said open distal end of said elongated container in a first direction and a second direction, said first direction being toward a biological sample contained in a vessel such that said open distal end of said elongated container becomes immersed in said biological sample and said second direction being away from said biological sample contained in a vessel such that said open distal end of said elongated container becomes withdrawn from said biological sample; and
(iii) a suction system attached to the open proximal end of said elongated container for drawing one or more volumes of said biological sample into said elongated container when said open distal end of said elongated container is immersed in said biological sample.

48. The apparatus of claim 47, wherein said suction system further draws a separation gas into said elongated container when said distal end of said elongated container is withdrawn from said biological sample.

49. The apparatus of claim 48, wherein said separation gas comprises air.

50. The apparatus of claim 47, further comprising: a computer system operating under the instruction of a software program, wherein said computer system controls the movement of said open distal end of said elongated container by said drive system as a function of time.

51. The apparatus of claim 47, further comprising: a computer system operating under the instruction of a software program, wherein said computer system controls the amount of suction pressure generated in said elongated container by said suction system as a function of time.

52. The apparatus of claim 51, further comprising: a computer system operating under the instruction of a software program, wherein said computer system controls the movement of said open distal end of said elongated container by said drive system as a function of time.

53. The apparatus of claim 47, wherein said suction system comprises a syringe.

54. The apparatus of claim 47, wherein said suction system comprises a pump.

55. The apparatus of claim 47, wherein said biological sample comprises a sample containing sperm.

56. The apparatus of claim 47, wherein said biological sample comprises a sample containing cells.

57. The apparatus of claim 47, wherein said elongated container has an undetermined length.

58. An apparatus, comprising:
(i) an elongated container having an open proximal end and an open distal end;
(ii) a drive system that causes movement of a vessel containing a biological sample; and
(iii) a suction system that causes a suction pressure in said elongated container, wherein said suction system is connected to said proximal end of said elongated container, and wherein said drive system is able to reversibly and repeatedly cause said distal end of said elongated container to contact and withdraw from said biological sample contained in said vessel.

59. An apparatus, comprising:
(i) a vessel containing a biological sample;
(ii) a vessel support for holding said vessel;
(iii) an elongated container of undetermined length having an open proximal end and an open distal end;
(iv) a holder for holding said elongated container near said open distal end such that said open distal end is positioned above said biological sample and pointed downward toward said biological sample;
(v) a drive system that causes movement of said vessel containing said biological sample toward and away from said open distal end of said elongated container; and
(vi) a suction system that causes a suction pressure in said elongated container, wherein said suction system is attached to said open proximal end of said elongated container.

60. An apparatus, comprising:
(i) a unibody elongated container having an open distal end and an open proximal open end;
(ii) a drive system that is capable of moving a vessel containing a biological sample in a first direction and a second direction, said first direction being toward said open distal end of said elongated container such that said open distal end of said elongated container becomes immersed in said biological sample and said second direction being away from said open distal end of said elongated container such that said open distal end of said elongated container becomes withdrawn from said biological sample; and
(iii) a suction system attached to the open proximal end of said elongated container for drawing one or more volumes of said biological sample into said elongated container when said open distal end of said elongated container is immersed in said biological sample.

61. The apparatus of claim 60, further comprising: a computer system operating under the instruction of a software program, wherein said computer system controls the movement of said vessel containing said biological sample by said drive system as a function of time.

62. The apparatus of claim 60, wherein said suction system further draws a separation gas into said elongated container when said distal end of said elongated container is withdrawn from said biological sample.

63. The apparatus of claim 61, wherein said separation gas comprises air.

64. The apparatus of claim 60, further comprising: a computer system operating under the instruction of a software program, wherein said computer system controls the amount of suction pressure generated in said elongated container by said suction system as a function of time.

65. The apparatus of claim 61, further comprising: a computer system operating under the instruction of a software program, wherein said computer system controls the movement of said vessel containing said biological sample by said drive system as a function of time.

66. The apparatus of claim 60, wherein said suction system comprises a syringe.

67. The apparatus of claim 60, wherein said suction system comprises a pump.

68. The apparatus of claim 60, wherein said biological sample comprises a sample containing sperm.

69. The apparatus of claim 60, wherein said biological sample comprises a sample containing cells.

70. The apparatus of claim 60, wherein said elongated container has an undetermined length.

71. An apparatus, comprising:
(i) a tube having a proximal end and a distal end;
(ii) an elongated container having an open proximal end and an open distal end;
(iii) a drive system that causes movement of said open distal end of said elongated container; and
(iv) a suction system that causes a suction pressure in said tube and said elongated container, wherein said suction system is connected to said proximal end of said tube, wherein said open proximal end of said elongated container is connected to said distal end of said tube, and wherein said drive system is able to reversibly and repeatedly cause said open distal end of said elongated container to contact and withdraw from a biological sample contained in a vessel.

72. An apparatus, comprising:
(i) a vessel containing a biological sample;
(ii) a vessel support for holding said vessel;
(iii) a tube of undetermined length having a proximal end and a distal end;
(iv) an elongated container having an open proximal end and an open distal end;
(v) a holder for holding said elongated container such that said open distal end of said straw is positioned above said biological sample and pointed downward toward said biological sample;
(vi) a drive system that causes movement of said open distal end of said elongated container toward and away from said biological sample contained in said vessel; and
(vii) a suction system that causes a suction pressure in said tube and said elongated container, wherein said open proximal end of said elongated container is connected to said distal end of said tube, and wherein said suction system is attached to said proximal end of said tube.

73. An apparatus, comprising:
(i) a tube having a distal end and a proximal open end;
(ii) an elongated container having an open proximal end and an open distal end, wherein said open proximal end of said elongated container is connected to said distal end of said tube;
(iii) a drive system that is capable of moving said open distal end of said elongated container in a first direction and a second direction, said first direction being toward a vessel containing a biological sample such that said open distal end of said elongated container becomes immersed in said biological sample and said second direction being away from said vessel containing said biological sample such that said open distal end of said elongated container becomes withdrawn from said biological sample; and
(iii) a suction system attached to said proximal end of said tube for drawing one or more volumes of said biological sample into said elongated container when said open distal end of said elongated container is immersed in said biological sample.

74. The apparatus of claim 73, wherein said elongated container comprises a straw.

75. The apparatus of claim 73, further comprising a multiport manifold, wherein said multiport manifold connects a plurality of elongated containers to said tube, such that said plurality of elongated containers become immersed and withdrawn from said biological sample together.

76. The apparatus of claim 73, wherein said suction system further draws a separation gas into said elongated container when said distal end of said elongated container is withdrawn from said biological sample.

77. The apparatus of claim 76, wherein said separation gas comprises air.

78. A method for drawing a biological sample into an elongated container, comprising:
a. providing an apparatus comprising (i) a vessel containing a biological sample, (ii) a vessel support for holding said vessel, an elongated container of undetermined length having an open proximal end and an open distal end, (iii) a holder for holding said elongated container near said open distal end of said elongated container such that said open distal end of said elongated container is positioned above said biological sample and pointed downward toward said biological sample, (iv) a drive system that causes movement of said holder and said open distal end of said elongated container toward and away from said biological sample, and (v) a suction system for causing a suction pressure in said elongated container;
b. causing said open distal end of said elongated container or said vessel containing said biological sample to move toward each other by said drive system such that said open distal end of said elongated container becomes immersed in said biological sample for a contacting period of time;
c. drawing a volume of said biological sample into said elongated container as a result of suction pressure generated in said elongated container by said suction system;
d. causing said open distal end of said elongated container or said vessel containing said biological sample to move away from each other by said drive system such that said open distal end of said elongated container is withdrawn from said biological sample for a withdrawn period of time; and
e. drawing a volume of a separation gas into said elongated container as a result of suction pressure generated in said elongated container by said suction system.

79. The method of claim 78, wherein said elongated container has a unibody construction and wherein said open proximal end of said elongated container is connected to said suction system.

80. The method of claim 78, wherein said apparatus further comprises a tube having a proximal end and a distal end, wherein said elongated container comprises a straw, wherein said open proximal end of said straw is connected to said distal end of said tube, and wherein said proximal end of said tube is connected to said suction system.

81. The method of claim 78, wherein said apparatus further comprises a tube having a proximal end and a distal end, wherein said elongated container comprises a straw, wherein said open proximal end of said straw is connected to said distal end of said tube, and wherein said proximal end of said tube is connected to said suction system.

82. The method of claim 78, further comprising:
f. repeating steps b. through e. at least once such that at least two volumes of said biological sample are drawn into said elongated container and separated by at least one volume of separation gas.

83. The method of claim 82, wherein said elongated container has a unibody construction and wherein said open proximal end of said elongated container is connected to said suction system.

84. The method of claim 82, wherein said apparatus further comprises a tube having a proximal end and a distal end, wherein said elongated container comprises a straw, wherein said open proximal end of said straw is connected to said distal end of said tube, and wherein said proximal end of said tube is connected to said suction system.

85. A method for drawing a biological sample into an elongated container, comprising:
a. providing an apparatus comprising an elongated container having an open proximal end and an open distal end.
b. causing said open distal end of said elongated container to contact a biological sample in a vessel for a contacting period of time;
c. drawing a volume of said biological sample into said elongated container by suction pressure generated by a suction system;
d. causing said open distal end of said elongated container to withdraw from said biological sample for a withdrawn period of time; and
e. drawing a volume of a separation gas into said elongated container by suction pressure generated by said suction system.

86. The method of claim 85, wherein said elongated container has a unibody construction of undetermined length and wherein said open proximal end of said elongated container is connected to said suction system.

87. The method of claim 85, wherein said apparatus further comprises a tube having a proximal end and a distal end, wherein said elongated container comprises a straw, wherein said open proximal end of said straw is connected to said distal end of said tube, and wherein said proximal end of said tube is connected to said suction system.

88. The method of claim 85, wherein said apparatus further comprises a computer system under the instruction of a software program, wherein said computer system controls the amount of suction pressure generated by said suction system as a function of time.

89. The method of claim 85, wherein said apparatus further comprises a drive system that causes said open distal end of said elongated container to move toward and away from said biological sample.

90. The method of claim 89, wherein said apparatus further comprises a computer system under the instruction of a software program, wherein said computer system controls the timing of contact and withdrawal of said open distal end of said elongated container with said biological sample by said drive system as a function of time.

91. The method of claim 89, wherein said drive system causes said open distal end of said elongated container to move toward and away from said vessel containing biological sample.

92. The method of claim 89, wherein said drive system causes said vessel containing said biological sample to move toward and away from said open distal end of said elongated container.

93. The method of claim 85, wherein said biological sample comprises a sample containing sperm.

94. The method of claim 85, wherein said biological sample comprises a sample containing cells.

95. The method of claim 85, wherein said biological sample is from a mammalian source.

96. The method of claim 85, wherein said separation gas comprises air.

97. The method of claim 85, further comprising the step of:
f. repeating steps (b) through (e) at least once such that at least two volumes of said biological sample are drawn into said elongated container and separated by at least one volume of separation gas.

98. The method of claim 97, wherein said elongated container has a unibody construction of undetermined length and wherein said open proximal end of said elongated container is connected to said suction system.

99. The method of claim 97, wherein said apparatus further comprises a tube having a proximal end and a distal end, wherein said elongated container comprises a straw, wherein said open proximal end of said straw is connected to said distal end of said tube, and wherein said proximal end of said tube is connected to said suction system.

100. The method of claim 97, wherein said volume of said biological sample varies with each round of steps b. through e.

101. The method of claim 97, wherein said apparatus further comprises a computer system under the instruction of a software program, wherein said computer system controls the amount of suction pressure generated by said suction system as a function of time.

102. The method of claim 97, wherein said apparatus further comprises a drive system that causes said open distal end of said elongated container to move toward and away from said biological sample.

103. The method of claim 102, wherein said apparatus further comprises a computer system under the instruction of a software program, wherein said computer system controls the timing of contact and withdrawal of said open distal end of said elongated container with said biological sample by said drive system as a function of time.

104. The method of claim 102, wherein said drive system causes said open distal end of said elongated container to move toward and away from said vessel containing biological sample.

105. The method of claim 102, wherein said drive system causes said vessel containing said biological sample to move toward and away from said open distal end of said elongated container.

106. The method of claim 98, further comprising the step of: cutting said elongated container to thereby release and separate a segment of said elongated container containing said volumes of biological sample from the remainder of said elongated container.

107. The method of claim 99, wherein after completing said steps, said straw is detached from said tube and is sealed at one or both ends.

108. The method of claim 104, wherein after completing said steps, said segment of said elongated container is sealed at one or both ends.

109. The method of claim 97, wherein said separation gas comprises air.
